(19) United States
(12) Patent Application Publication
(10) Pub. No.: US 2025/0088987 A1
(43) Pub. Date: Mar. 13, 2025

(54) SYNCHRONIZING IMPLANTABLE ELECTRONICS

(71) Applicant: Phantom Neuro Inc., Austin, TX (US)

(72) Inventors: Michele Adriani, Cambridge (GB); Martin Pierre Émile Laporte, Mazerolles (FR); Joseph Joyce, Peterborough (GB); Robert Rudolf, Cambridge (GB); Isabella Miele, Cambridge (GB); Bogdan-Alexandru Cociu, Cambridge (GB)

(21) Appl. No.: 18/825,206

(22) Filed: Sep. 5, 2024

Related U.S. Application Data
(60) Provisional application No. 63/581,204, filed on Sep. 7, 2023.

(51) Int. Cl. H04W 56/00; H04W 72/0446

(52) U.S. Cl. CPC H04W 56/0015; H04W 56/0065; H04W 72/0446

(57) ABSTRACT

Systems and methods for wireless synchronization in an implantable system include receiving, by a first computing device, a first synchronization signal; recording, by the first computing device, a first timestamp based on a time of receipt of the first synchronization signal; receiving, by the first computing device, a synchronization request comprising a second timestamp; determining, by the first computing device, a synchronized time offset based on the first timestamp and the second timestamp; transmitting, by the first computing device, a second synchronization signal; generating, by the first computing device, a synchronization message comprising a third timestamp based on a time of transmission of the second synchronization signal and the synchronized time offset; queuing, by the first computing device, the synchronization message during a non-accessible transmission time slot; and wirelessly transmitting, by the first computing device, the synchronization message to a second computing device during an available signal transmission time slot.

### CROSS-REFERENCE TO RELATED APPLICATIONS

This application claims the benefit under 35 U.S.C. § 119(e) of U.S. Provisional Application No. 63/581,204, entitled “Synchronizing Implantable Electronics,” filed Sep. 7, 2023, which is incorporated herein by reference in its entirety.

### TECHNICAL FIELD

This disclosure relates to synchronization of electronic devices and system, and more particularly to synchronization of implantable systems, implantable devices, and related methods of controlling a prosthesis.

### BACKGROUND

There are currently hundreds of millions of individuals worldwide with mobility impairments resulting from aging and/or physical disabilities. Robotic limb prostheses, active orthotics, and exoskeletons can help replace and/or augment the motor function of amputated or impaired biological limbs and allow users to perform daily activities that require the use of motorized orthopedic technologies, including those described in WO 2021/242775 A1, which is incorporated by reference in its entirety. However, the control of these wearable robotic devices is extremely difficult and often considered one of the leading challenges to real-world deployment. Thus, improvements in control of wearable robotic devices are continually sought.

### SUMMARY

In general, this disclosure relates to synchronization of electronic devices and systems and in particular implantable systems, implantable devices, and related methods. Such implantable devices can be used for the wireless detection and transmission of EMG signals generated by one or more muscles of a subject. When more than one implantable device is used in an implantable system, the implantable system can acquire time-series data if the system is synchronized to a common time reference; however, generally, electronic devices have unknown time references and clock drifts posing a significant challenge to acquiring synchronized data. An electronic computing device receives a synchronization signal and records the time of receipt of the synchronization signal. The computing device receives a synchronization request from an external computing device including a timestamp corresponding to the time of transmission of the synchronization signal according to the clock of the external computing device. The first computing device determines a synchronized time offset based on the times of transmission and reception of the synchronization signal. The first computing device can forward the synchronized time to additional computing devices by waiting for a fresh synchronization signal and then sending a synchronization message in the next available transmission time slot.

Certain implementations of the systems and methods of this disclosure can provide technical advantages. Multiple electronic computing devices can be synchronized to a common time reference. The synchronization can occur over a bi-directional communications link using a time-domain multiple access protocol. Devices can be synchronized over a variety of communications links (e.g., short range wireless communications and near field magnetic induction communications). The systems and methods of this disclosure can also be applied to synchronizing disparate processors within a single device that may have unsynchronized clocks.

In an example implementation, a method for wireless synchronization in an implantable system includes receiving, by a first computing device, a first synchronization signal; recording, by the first computing device, a first timestamp based on a time of receipt of the first synchronization signal; receiving, by the first computing device, a synchronization request comprising a second timestamp; determining, by the first computing device, a synchronized time offset based on the first timestamp and the second timestamp; transmitting, by the first computing device, a second synchronization signal; generating, by the first computing device, a synchronization message including a third timestamp based on a time of transmission of the second synchronization signal and the synchronized time offset; queuing, by the first computing device, the synchronization message during a non-accessible transmission time slot; and wirelessly transmitting, by the first computing device, the synchronization message to a second computing device during an available signal transmission time slot.

An aspect combinable with the example implementation, includes receiving, by the first computing device, a synchronization acknowledgment signal from the second computing device.

Another aspect combinable with any of the previous aspects includes receiving, by the second computing device, the second synchronization signal; recording, by the second computing device, a fourth timestamp based on a time of receipt of the second synchronization signal; receiving, by the second computing device, the synchronization message; and determining, by the second computing device, a second synchronized time offset based on the synchronization message and the fourth timestamp.

In another aspect combinable with any of the previous aspects, the synchronization message includes a data acquisition start time.

Another aspect combinable with any of the previous aspects includes starting, by the second computing device and based on the second synchronized time offset, synchronized data transmission at the data acquisition start time.

Another aspect combinable with any of the previous aspects includes receiving, by the first computing device, a synchronized data transmission from the second computing device.

Another aspect combinable with any of the previous aspects includes transmitting, by the first computing device, the synchronized data transmission to a third computing device.

In another aspect combinable with any of the previous aspects, wirelessly transmitting, by the first computing device, includes wirelessly transmitting, by the first computing device, the synchronization message via a near field magnetic induction communications link.

In another aspect combinable with any of the previous aspects, receiving, by the first computing device, a synchronization request comprises receiving, by the first computing device, the synchronization request from a third computing device.

In another aspect combinable with any of the previous aspects, receiving, by the first computing device, a synchronization request further comprises receiving, by the first computing device, the synchronization request via a short range communications link.

Another aspect combinable with any of the previous aspects includes configuring a short range communications link between the first computing device and a third computing device to generate the first synchronization signal during an expected time window; and transmitting the first synchronization signal by a third computing device.

Another aspect combinable with any of the previous aspects includes determining, by the third computing device, that the first synchronization signal did not occur during the expected time window; generating, by the third computing device, a virtual synchronization signal in place of the first synchronization signal; and transmitting, by the third computing device, the virtual synchronization signal to the first computing device during the expected time window.

In another aspect combinable with any of the previous aspects, the expected time window includes a transmission time slot of the short range communications link.

In another aspect combinable with any of the previous aspects, determining the synchronized time offset includes storing, into memory of the first computing device, a time delay based on the first timestamp and the second timestamp; and determining, by the first computing device, the synchronized time offset based on the time delay and previously stored time delays.

Another aspect combinable with any of the previous aspects includes receiving, by the first computing device, a fresh synchronization signal after receiving the synchronization request, wherein queuing the synchronization message occurs after receiving the fresh synchronization request, and wherein wirelessly transmitting the synchronization message occurs in the next available signal transmission time slot following receiving the fresh synchronization signal.

In another example implementation, a method of controlling a prosthesis includes receiving, by two or more wearable devices a first synchronization signal from an external device, each wearable device associated with an implantable device; by each wearable device recording a first timestamp based on a time of receipt of the first synchronization signal; receiving a synchronization request from the external device comprising a second timestamp; determining a synchronized time offset based on the first timestamp and the second timestamp; transmitting a second synchronization signal to the associated implantable device; generating a synchronization message comprising a third timestamp based on a time of transmission of the second synchronization signal and the synchronized time offset; queuing the synchronization message during a non-accessible transmission time slot; wirelessly transmitting the synchronization message to a second computing device during an available signal transmission time slot; wirelessly receiving synchronized EMG signals from the implantable devices, the EMG signals being synchronized based on the synchronization message; wirelessly transmitting the synchronized EMG signals to the external device; processing the synchronized EMG signals using one or more machine learning classifiers; and generating a control output for the prosthesis based on the processing.

In an aspect combinable with the example implementations, wirelessly transmitting the synchronization message includes wirelessly transmitting via a near field magnetic induction communications link.

In another aspect combinable with any of the previous aspects, receiving a synchronization request from an external device includes receiving a synchronization request from an external device via a wireless short range communications link.

Another aspect combinable with any of the previous aspects includes at each wearable device, receiving a synchronization acknowledgment message from the implantable device.

Another aspect combinable with any of the previous aspects includes by each wearable device, transmitting the synchronization acknowledgement message to the external device.

In another aspect combinable with any of the previous aspects, the control output comprises one or more of a continuous joint angle, a movement, or a discrete gesture.

In another aspect combinable with any of the previous aspects, the one or more machine learning classifiers are one or more of a discrete classifier or a continuous classifier.

In another aspect combinable with any of the previous aspects, the discrete classifier is a logistic regression classifier.

In another aspect combinable with any of the previous aspects, the one or more machine learning classifiers are trained machine learning classifiers.

Another aspect combinable with any of the previous aspects includes training the machine learning classifier, where training the machine learning classifier includes receiving, via a processor, sensor data including one or more of the EMG signal or a motion signal corresponding to a movement of a subject; and training the machine learning classifier, via the processor, to generate one or more hyperparameters configured to be stored on the processor and configured to control the prosthesis.

Another aspect combinable with any of the previous aspects includes configuring a short range communications link between the external device and the wearable devices to generate the first synchronization signal during an expected time window; and transmitting the first synchronization signal by the external device.

Another aspect combinable with any of the previous aspects includes determining, by the external device, that the first synchronization signal did not occur during the expected time window; generating, by the external device, a virtual synchronization signal in place of the first synchronization signal; and transmitting, by the external computing device, the virtual synchronization signal to the wearable devices during the expected time window.

In another aspect combinable with any of the previous aspects, the expected time window includes a transmission time slot of the short range communications link.

In another aspect combinable with any of the previous aspects, determining the synchronized time offset includes storing, into memory of each wearable device, a time delay based on the first timestamp and the second timestamp; and determining, by the wearable device, the synchronized time offset based on the time delay and previously stored time delays.

Another aspect combinable with any of the previous aspects includes receiving, by each wearable device, a fresh synchronization signal after receiving the synchronization request, wherein queuing the synchronization message occurs after receiving the fresh synchronization request, and wherein wirelessly transmitting the synchronization message occurs in the next available signal transmission time slot following receiving the fresh synchronization signal.

In another example implementation, an implantable system includes one or more implantable devices including an implantable substrate including a sensor configured to detect and transmit an electromyography (EMG) signal generated by a muscle of a subject, the implantable substrate is in contact with the muscle of the subject; and a first processor operatively coupled with the implantable substrate and configured to perform operations including receiving a synchronization signal; recording a timestamp based on a time of receipt of the synchronization signal; receiving a synchronization message; and determining a synchronized time offset based on the synchronization message and the timestamp; receiving the EMG signal from the sensor; starting transmission of the EMG signal based on the synchronization message; and wirelessly transmitting the EMG signal; one or more wearable devices configured to be attached to the subject, the one or more wearable devices includes a second processor configured to perform operations including receiving a second synchronization signal from an external device; recording a second timestamp based on a time of receipt of the second synchronization signal; receiving a synchronization request comprising a third timestamp; determining a second synchronized time offset based on the second timestamp and the third timestamp; transmitting the synchronization signal; generating the synchronization message comprising a fourth timestamp based on a time of transmission of the synchronization signal and the second synchronized time offset; queuing the synchronization message during a non-accessible transmission time slot; and wirelessly transmitting the synchronization message to the first processor during an available signal transmission time slot; a self-contained battery; and a power transmitter configured to wirelessly transmit energy to the one or more implantable devices via an inductive magnetic field.

In an aspect combinable with the example implementation, the first processor is configured to perform operations including wirelessly transmitting a synchronization acknowledgement signal; and the second processor is configured to perform operations including wirelessly receiving the synchronization acknowledgement from the first processor; and wirelessly transmitting the synchronization acknowledgement signal to the external device.

In another aspect combinable with any of the previous aspects, the synchronization message includes a time at which to start transmission of the EMG signal.

In another aspect combinable with any of the previous aspects, the synchronization signal includes a radio available signal.

In another aspect combinable with any of the previous aspects, the second processor is configured to perform receiving the synchronization request from the external device and transmitting the EMG signal to the external device via a short range communications link.

In another aspect combinable with any of the previous aspects, the second processor is configured to transmit the synchronization message to the first processor and receive the EMG signal from the first processor via a near field magnetic induction communications link.

In another aspect combinable with any of the previous aspects, the implantable system is used in a prosthetic limb.

In another aspect combinable with any of the previous aspects, the implantable system is used in an orthotic.

In another aspect combinable with any of the previous aspects, the implantable system is used in an exoskeleton.

In another aspect combinable with any of the previous aspects, the sensor is an EMG sensor.

Another aspect combinable with any of the previous aspects includes a motion sensor.

In another aspect combinable with any of the previous aspects, the EMG sensor and the motion sensor are configured to be inductively powered by an external power source.

Another aspect combinable with any of the previous aspects includes a second EMG sensor configured to detect and transmit the EMG signal generated by a second muscle of the one or more muscles of the subject, the sensor is a first sensor configured to detect and transmit the EMG signal generated by a first muscle of the one or more muscles of the subject.

In another aspect combinable with any of the previous aspects, each of the first muscle and the second muscle is a skeletal muscle or a portion thereof.

In another aspect combinable with any of the previous aspects, the one or more implantable devices are coated with or encapsulated within a biocompatible material and/or a bioinert material.

In another aspect combinable with any of the previous aspects, the biocompatible and/or bioinert materials includes silicone.

In another aspect combinable with any of the previous aspects, the biocompatible and/or bioinert materials includes at least one of a silicone material, a ceramic material, an epoxy material, a fabric, a cellular scaffold, an acellular scaffold, a plastic material, a metallic material, a carbon derivative, a synthetic biological tissue, a polymer, a meshed material.

In another aspect combinable with any of the previous aspects, the first processor is configured to wirelessly receive the EMG signal from the sensor.

In another aspect combinable with any of the previous aspects, the processor is configured to wirelessly receive the EMG signal from the sensor.

In another aspect combinable with any of the previous aspects, the self-contained battery is rechargeable.

In another aspect combinable with any of the previous aspects, the one or more implantable devices include a power receiver.

In another aspect combinable with any of the previous aspects, the power transmitter further includes an amplifier configured to amplify an electrical signal to generate the inductive magnetic field.

In another aspect combinable with any of the previous aspects, the one or more wearable devices further include a decoder configured to wirelessly receive and decode the EMG signal.

In another aspect combinable with any of the previous aspects, the sensor of the one or more implantable devices further includes one or more electrodes embedded within the implantable substrate.

In another aspect combinable with any of the previous aspects, the one or more electrodes include sensing electrodes.

In another aspect combinable with any of the previous aspects, the sensor includes two or more electrodes that are spaced equidistantly from a center of each of the two or more electrodes.

In another aspect combinable with any of the previous aspects, the implantable substrate has a first surface and a second surface on the opposite side of the first surface, and wherein the sensor is embedded within the first surface and one or more reference electrodes are embedded within the second surface.

In another aspect combinable with any of the previous aspects, an analogue switch or multiplexer operable to reconfigure the one or more reference electrodes in situ.

In another aspect combinable with any of the previous aspects, the implantable substrate is configured to wrap around a circumference of the one or more muscles of the subject when implanted.

In another aspect combinable with any of the previous aspects, the implantable substrate is a flat, elongated strip.

In another aspect combinable with any of the previous aspects, the implantable substrate is flexible and configured to wrap around the one or more muscles of the subject.

In another aspect combinable with any of the previous aspects, the one or more implantable devices further include a case connected to a connector of the implantable substrate, the case configured to house one or more electronic components.

In another aspect combinable with any of the previous aspects, the case is composed of a biocompatible material and/or a bioinert material.

The details of one or more implementations of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an example distributed sensor system.

FIG. 2A is a flow chart of an example method for synchronizing computing devices.

FIG. 2B is a flow chart of another example method for synchronizing computing devices.

FIGS. 3A-3B are example timing diagrams according to the method of FIG. 2A.

FIG. 4 is a schematic diagram showing an example of an implantable system, as described herein.

FIG. 5 is a block diagram of an example implantable system.

FIG. 6 is a timing diagram of an example synchronization procedure for the implantable system of FIG. 5.

FIG. 7 is a perspective view of an example implantable device.

FIG. 8A is a perspective view of an implantable substrate of the implantable device of FIG. 2.

FIG. 8B is an enlarged view of the exposed portion of an electrode of the implantable substrate of FIG. 8A.

FIG. 9A is an enlarged, perspective view of an electronic module of the implantable device of FIG. 7.

FIG. 9B is an enlarged, side view of an example connector of the electronic module of FIG. 9A located at a first position.

FIG. 9C is an enlarged, side view of an example connector of the electronic module of FIG. 9A located at a second position.

FIG. 10A is a perspective view of an example implantable device.

FIG. 10B is a perspective view of an example implantable substrate.

FIG. 10C is a perspective view of an example feedthrough connector.

FIG. 10D is a perspective view of an example feedthrough connector.

FIG. 11A is a perspective view of a wearable device.

FIG. 11B is a top view of the wearable device of FIG. 11A.

FIG. 11C is a perspective, cross-sectional view of the wearable device of FIG. 11A.

FIG. 12A is a perspective view of a circular wearable device.

FIG. 12B is a perspective view of a rectangular wearable device.

FIG. 13 is a schematic diagram depicting examples of angular misalignment, lateral misalignment, and depth of the implantable substrate with respect to a wearable device.

### DETAILED DESCRIPTION

### Device Synchronization

Distributed sensor systems (e.g., Internet of Things (IoT) sensor systems) typically do not inherently have a shared time reference between connected devices of the system. For example, each device can have a unique and unknown time zero based on when the device was powered on, and each device can have a unique and unknown clock drift. To collect time synchronized data from multiple devices, a time synchronization routine can be used to generate a shared time reference. The shared time reference can be used to start sampling data on multiple devices at the same moment in time.

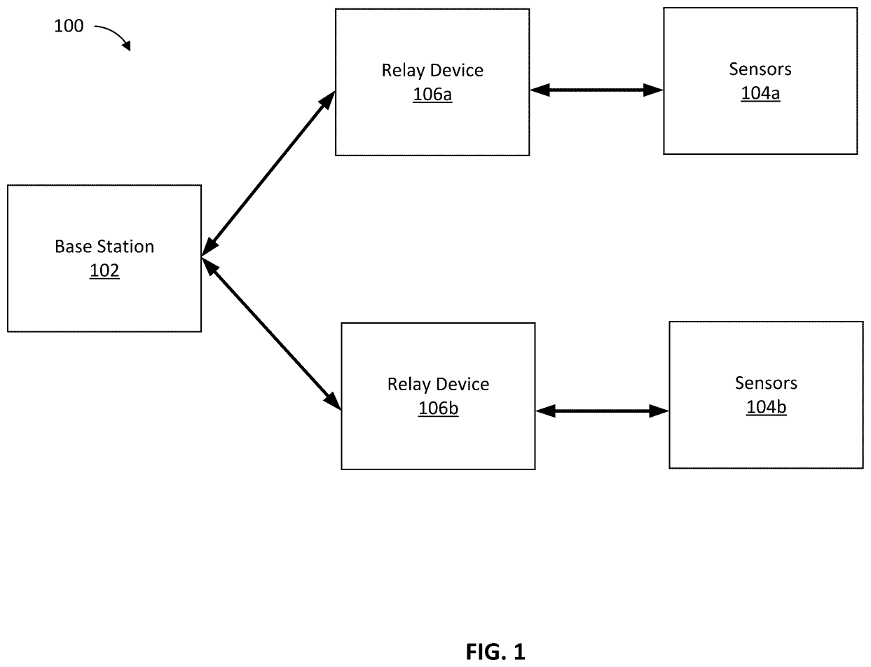

FIG. 1 is a block diagram of an example distributed sensor system 100. The distributed sensor system 100 includes a base station 102, sensors 104a-b, and relay devices 106a-b. Each of the devices of the distributed sensor system 100 include at least one processor, memory storing software and/or firmware to control the device, and a communications interface. The distributed sensor system 100 is configured such that each of the devices can wirelessly communicate with at least one other device of the system. Each device is configured for two-way communication over a short range wireless communications link (e.g., Bluetooth®, Bluetooth® Low Energy, near field magnetic induction (NFMI)). For example, sensor 104a can wirelessly communicate with relay device 106a, relay device 106a can communicate with both the sensor 104a and the base station 102, and the base station 102 can communicate with both relay devices 106a-b. Although distributed sensor system 100 is shown to include two relay devices and two sensors, any number of relay devices and sensors can be included in the system.

Base station 102 can be a personal computing device. For example, the base station can be a desktop or laptop computer. In some implementations, the base station 102 is a smartphone or a tablet.

Relay devices 106a-b are configured to communicate with the base station 102 and the sensors 104a-b. The relay devices 106a-b can be, for example, a wearable device or a mobile computing device. The relay devices 106a-b can be configured to communicate with the base station using a short range wireless communications link (e.g., Bluetooth® or Bluetooth® Low Energy). Simultaneously, the relay devices 106a-b can be configured to communicate with the sensors 104a-b using a near field magnetic induction communications link. In some implementations, the relay devices 106a-b can be configured to communicate with the sensors 104a-b using a short range wireless communications link.

Sensors 104a-b are configured to acquire measurement data and transmit the data to the relay devices 106a-b. For example, the sensors 104a-b can be implantable sensors that measure electromyography (EMG) signals generated by skeletal muscles. The EMG data can be transmitted to the relay devices 106a-b. The relay devices 106a-b can relay the EMG data to the base station 102 for further processing.

The communications interfaces of the base station 102 and the relay devices 106a-b can be configured to use a time-domain multiple access (TDMA) communications protocol to allow communication with multiple devices in the distributed sensor system 100. A TDMA communications protocol allows multiple devices to communicate over a shared frequency channel by dividing the radio signal of the channel into frames. A frame is further subdivided into time slots. Each connected device is assigned a time slot within the frame during which to transmit and receive signals. For example, the base station 102 and the relay devices 106a-b can be configured to communicate over the same radio channel. The radio channel can be divided into frames of a specified duration (e.g., 20 ms). Each frame is divided into two time slots, one corresponding with each of the relay devices 106a-b. During the first time slot relay device 106a can transmit and receive messages with the base station 102. During the second time slot, relay device 106b can transmit and receive messages with the base station 102. The base station 102 can transmit a synchronization or radio available signal to each of the relay devices 106a-b. The synchronization or radio available signal provides a common reference point for each of the devices; however, the synchronization or radio available signal does not provide a shared time between the devices (e.g., each device can have a different clock time corresponding to the occurrence of the synchronization signal).

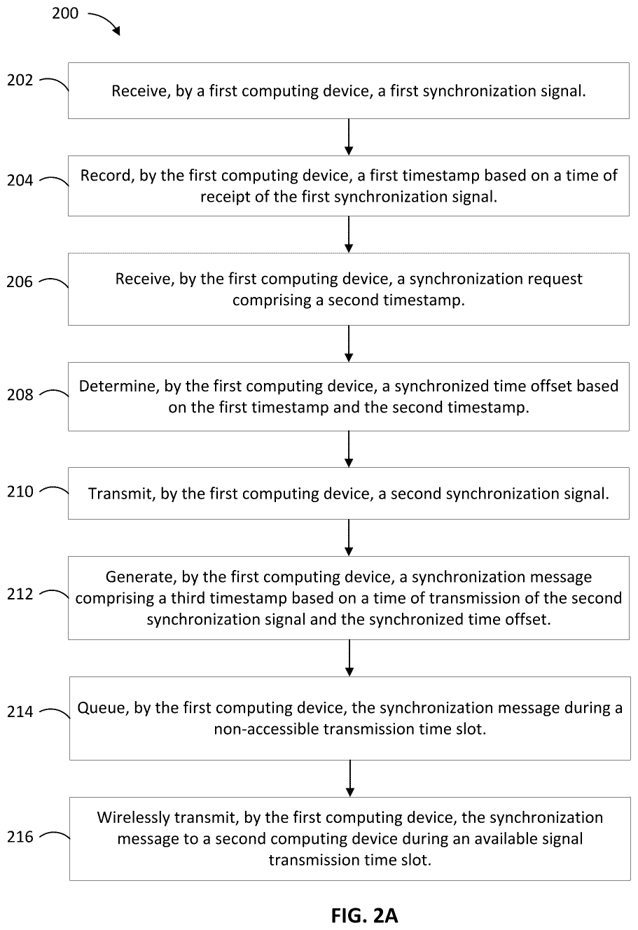

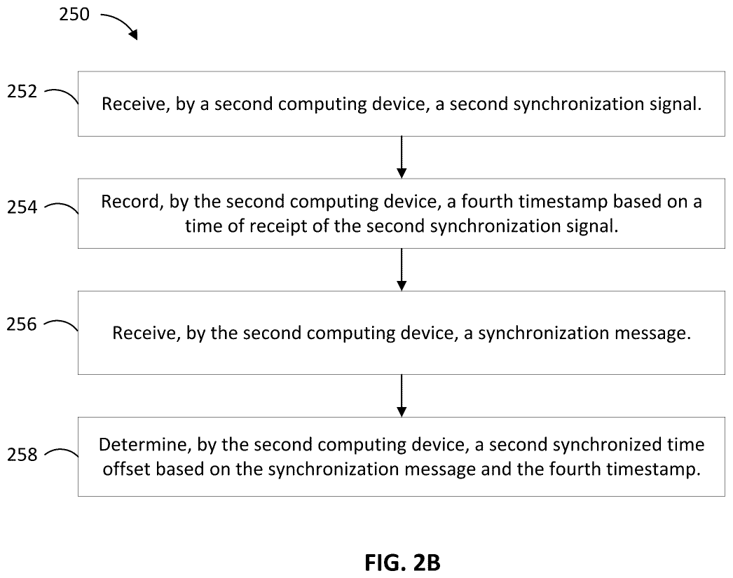

FIG. 2A is a flow chart of an example method 200 for synchronizing a distributed sensor system (e.g., the distributed sensor system 100). The method 200 synchronizes the devices of the distributed sensor system by establishing a common time reference among all of the devices. After establishing the common time reference, synchronized data acquisition can begin at a specified time.

A first computing device receives a first synchronization signal (step 202). The first computing device can be, for example, one of the relay devices 106a-b. The first computing device can receive the first synchronization signal from an external computing device, for example, base station 102. The first synchronization signal can be a part of a periodically provided synchronization signal. For example, the external computing device can provide a radio available synchronization signal over a short range wireless communications link to other paired devices. The external computing device can configure the short range communications link to provide the synchronization signal during an expected time window. The external computing device can monitor the provided signals, and when the external computing device determines that a synchronization signal is missing, the external computing device can generate a virtual synchronization signal. The external computing device can transmit the virtual signal during the expected time window, so that the first computing device receives the first synchronization signal when it is expected. The expected time window can be, for example, a TDMA time slot available for transmission to the first computing device. In some implementations, the first computing device generates the synchronization signal internally.

The first computing device records a timestamp t₁ corresponding to a time of receipt of the first synchronization signal (step 204). The first computing device can be configured to record the timestamp t₁ of the most recent synchronization signal received according to the clock time of the first computing device T₁. By so doing, the first computing device maintains a synchronization timestamp. In some implementations, the first computing device can generate a simulated synchronization signal based on a period of the synchronization signal. The simulated synchronization signal can be used when a real synchronization signal is not received at an expected time. For example, if the synchronization signal has a period of 20 ms, a simulated synchronization signal can be generated by adding the period (20 ms) to the most recent synchronization timestamp.

The first computing device receives a synchronization request (step 206). The synchronization request can be received from the external computing device (e.g., the base station 102). The synchronization request includes a second timestamp t₂. The second timestamp corresponds to the time at which the synchronization signal was transmitted from the external computing device according to the clock of the external computing device T_{E}.

The first computing device determines a synchronized time offset AT based on the first timestamp t₁ and the second timestamp t₂ (step 208). The first timestamp t₁ corresponds to the time at which the synchronization signal was received by the first computing device according to the clock of the first computing device T₁. The second timestamp t₂ corresponds to the time at which the synchronization signal was transmitted by the external computing device according to the clock of the external computing device T_{E}. The synchronized time offset At can be determined by, for example, taking the difference between the second timestamp and the first timestamp, e.g., Δt=t₂−t₁. Accordingly, the clock of the first computing device can be synchronized with the clock of the external computing device by adding the synchronized time offset to the current clock time of the first computing device (e.g., T_{E} T₁+Δt).

The first computing device can relay the synchronization to additional computing devices in the distributed sensor system. For example, the relay device 106a can be synchronized with the base station 102 by applying steps 202-208 of the method 200. The relay device 106a can then forward the synchronization to the sensor 104a. The relay device 106a can be synchronized with the sensor 104a by applying steps 210-216 of the method 200.

The first computing device transmits a second synchronization signal (step 210). The second synchronization signal can be transmitted via a separate communications link than the first synchronization signal. For example, the second synchronization signal can be transmitted via an NFMI communications link whereas the first synchronization signal can be transmitted via a short range wireless communication link (e.g., Bluetooth®). In some implementations, the first and second synchronization signals are transmitted over the same wireless communications link.

The first computing device generates a synchronization message comprising a third timestamp t₃ based on the time of transmission of the second synchronization signal, T_{1,transmit}, and the synchronized time offset (e.g., t₃=T_{1,transmit}+Δt), where T_{1,transmit} is the clock time of the first computing device at the time of transmission (step 212). The third timestamp t₃ is the synchronized time of transmission of the second synchronization signal (e.g., t₃ is the time of transmission of the second synchronization signal according to the clock time of the external computing device at the time of transmission).

The first computing device queues the synchronization message during a non-accessible transmission time slot (step 214). Queuing the synchronization message during a non-accessible transmission time slot prepares the message to be transmitted during the next accessible transmission time slot. For example, the first computing device can wait for a fresh synchronization signal after receiving the synchronization request. After receiving the fresh synchronization signal, the first computing device queues the synchronization message to be transmitted.

The first computing device wirelessly transmits the synchronization message to a second computing device during an available signal transmission time slot (step 216). For example, the first computing device transmits the synchronization message during the next available time slot after receiving the fresh synchronization signal. In this manner, the synchronization message is reliably transmitted before a subsequent synchronization signal. If the synchronization message is not transmitted during the synchronization period corresponding to the third timestamp t₃, then the synchronized time can have an error equal to the synchronization period.

In some implementations, the synchronization message includes a data acquisition start time at which the second computing device should begin acquiring data. The data acquisition start time is given relative to the shared, synchronized reference time. For example, the data acquisition start time can be the clock time of the external computing device at the time the synchronization request was sent to the first computing device plus a time delay (e.g., 3 seconds).

In some implementations, the first computing device receives a synchronization acknowledgment signal from the second computing device. The first computing device can also relay the synchronization acknowledgment message to the external computing device.

FIG. 2B shows a flow chart of an example method 250 for synchronizing the time of the second computing device with the first computing device. The second computing device receives the second synchronization signal (step 252). The second computing device records a fourth timestamp t₄ based on a time of receipt of the second synchronization signal by the second computing device. The fourth timestamp t₄ is the time of receipt according to the clock time of the second computing device, T2.

The second computing device receives the synchronization message transmitted by the first computing device (step 254). The synchronization message includes the third timestamp corresponding to the time of transmission of the synchronization signal according to the synchronized time reference.

The second computing device determines a second synchronized time offset Δt₂ based on the synchronization message and the fourth timestamp. For example, the second synchronized time offset can be determined by taking the difference between the third timestamp and the fourth timestamp, Δt₂=t₃−t₄. The clock time of the second computing device can be synchronized with both the first computing device and the external computing device by adding the second synchronized time offset to the current clock time of the second computing device (e.g., T_{E}−T₂+Δt₂). In this manner, the first, second, and external computing devices share a time reference synchronized with the clock of the external computing device.

The methods 200 and 250 can be repeated to synchronize any number of devices to a common reference time. In some implementations, the methods 200 and 250 can be used to synchronize two or more processors within a single computing device that do not share a clock time.

In implementations where the synchronization method includes a data acquisition start time, the second computing device can begin transmission of data at the specified data acquisition start time. When more than one sensor or data acquisition node is synchronized to the common reference time, the data acquisition from each sensor or data acquisition node can begin at the same moment in time.

The first computing device receives the data transmission from the second computing device. The first computing device can relay the data transmission to the external computing device. In some implementation, the external computing device is configured to process the transmitted data using, for example, a trained machine learning model.

In some implementations, the methods 200 and/or 250 are iteratively performed to mitigate synchronization errors arising from noisy communications channels, delayed synchronization messages, or lost packets. For example, the synchronization procedure can be repeated 5 times in a row before the start of data acquisition. The synchronized computing devices can keep a record of the 5 most recent synchronizations and use the synchronized time offset determined by the majority of the recent synchronizations. For example, the computing devices can store a synchronization time delay based on the first timestamp and the second timestamp and determine the synchronized time offset based on the synchronized time delay and previously stored synchronized time delays. The time delay for the data acquisition start time can be determined based on an anticipated time to complete the specified number of synchronizations.

In some implementations, the synchronization between computing devices can be periodically refreshed. Refreshing the synchronization can be accomplished by performing the methods 200 and 250 when desired.

In implementations synchronizing multiple sets of relay devices and sensors with a single base station or external computing device, the synchronization methods 200 and 250 can be performed with each pair of relay devices and sensors in series. For example, referring to FIG. 1, the base station 102 can first synchronize the relay device 106a and the sensor 104a by applying methods 200 and 250. Following the synchronization of the relay device 106a and the sensor 104a, the base station 102 can synchronize the relay device 106b and the sensor 104b. The application of the methods 200 and 250 will result in both relay devices 106a-b and both sensors 104a-b being synchronized to the time of the base station 102.

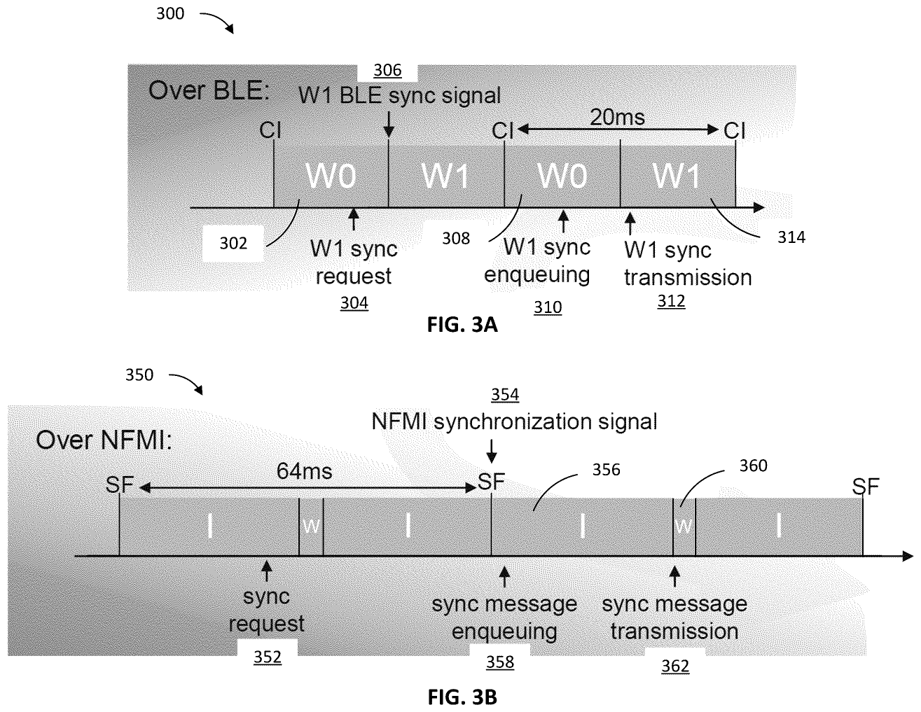

FIG. 3A is an example timing diagram 300 for synchronization of a computing device using a short range wireless communications link (e.g., Bluetooth® Low Energy, BLE). In this example, the computing device is linked to two other computing devices (W0 and W1). The connection interval (CI) is 20 ms long and is divided between the two paired computing devices W0 and W1. During a transmission time slot 302 for W0, the computing device receives a synchronization request 304 for W1. The computing device waits for a fresh synchronization signal 306 to occur. During the next non-accessible transmission time slot 308 for W1 (e.g., the next transmission time slot for W0), the computing device queues 310 a synchronization message for W1. The computing device transmits 312 the synchronization message during the next accessible time slot 314 for W1.

FIG. 3B is an example timing diagram 350 for synchronization of a computing device using an NFMI communications link. In this example, the NFMI signal is divided into super frames (SF) with a duration of 64 ms. The computing device receives a synchronization request 352. The computing device waits for a fresh synchronization signal 354. During the subsequent non-accessible time slot 356, the computing device queues 358 a synchronization message. In the next available time slot 360, the computing device transmits 362 the synchronization message.

### Implantable System

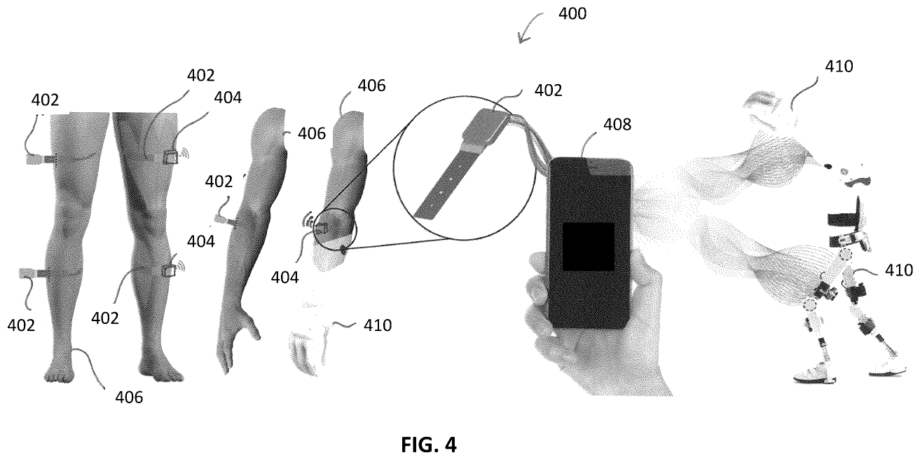

FIG. 4 is a schematic diagram showing an example of an implantable system 400 that can be used to control an orthopedic device (e.g., a prosthesis and/or an exoskeleton) via the detection of EMG signals generated by one or more muscles of a subject 406. The implantable system 400 includes one or more implantable devices 402 that can be implanted in a pocket formed within the subcutaneous or subadipose or subfascial anatomical planes in a subject 406, a wearable device 404 that can be removably secured to a limb of the subject 406, an external processing unit (EPU) 408, and a peripheral device 410.

The implantable device 402 includes one or more EMG sensors that can detect EMG signals generated by one or more muscles of the subject 406. The wearable device 404 can wirelessly connect to, power, and recharge the implantable device 402 when placed on a skin surface of the subject, near the vicinity where the electronic module 414 of the implantable device 402 is located. In this manner, the wearable device 404 can wirelessly transmit data (e.g., EMG data, motion data, a configuration parameter, a status parameter, and/or other types of sensor data) to and from the implantable device 402 via a wireless induction link, or other type of wireless communication system. In some implementations, the wireless communication system includes, but is not limited to, a galvanic communication system, a capacitive communication system, a radio frequency (RF) communication system, an inductive communication system, an ultrasound communication system, an optical communication system, and a molecular communication system. From the wearable device 404, the data is configured to be transmitted either via wireless link, or a hardwired link to the EPU 408. The EPU 408 can be a smartphone or other portable processing unit. The EPU 408 can be a part of the wearable device 404 or the peripheral device 410. In some implementations, the EPU 408 may be a central processing unit (CPU), a graphics processing unit (GPU), neural processing unit (NPU) or a neuromorphic processor, or any other specialized processor for running machine learning algorithms. On the EPU 408, the data is received from the wearable device 404 where it is processed and analyzed by one or more algorithms (e.g., a machine learning classifier or model, time-series signal processing).

As shown in FIG. 4, the implantable system can include two or more implantable substrates 402 that can work as a cohesive system where the two or more implantable substrate 402 can wirelessly connect to, be powered by, and be recharged by two or more wearable devices 404. In this manner, the wearable devices 404 can wirelessly transmit data (e.g., EMG data, motion data, and/or other types of sensor data) to and from the two or more implantable substrates 402 via a wireless induction link, or other type of wireless communication system. In some implementations, all of the wearable devices 404 of the system can wirelessly transmit data to and from all of the implantable substrates 402 of the system. In some implementations, a specific wearable device 404 in an implantable system 400 including two or more wearable devices 404 can be configured to wirelessly transmit data only to and from a specific implantable device 402 from all of the implantable devices 402 of the implantable system 400. For example, in some implementations, an implantable system includes first, second, and third wearable devices and first, second, and third implantable substrates, and the first wearable device is configured to wirelessly transmit data only to and from a first implantable device. From the wearable devices 404, the data from all of the implantable substrates 402 present in the implantable system 400 can be configured to be transmitted either via wireless link, or a hardwired link to the EPU 408 where it can be processed and analyzed by one or more algorithms (e.g., a machine learning classifier or model). In some implementations, the two or more implantable substrates are in contact with two or more different muscles. In some implementations, the two or more implantable substrates are in contact with two or more different portions of the same muscle.

Before an algorithm can be used to process and classify incoming data, it must be trained. In some implementations, training of the algorithm initially takes place on the EPU 408 (e.g., on a smartphone) or on the cloud. Once the algorithm has been trained, the trained algorithm is configured to process the input data in real-time, to control the peripheral device 410. The trained algorithm is configured to receive data and produce control outputs for the peripheral device 410, such as, but not limited to, continuous joint angles, discrete gestures, or other control parameters. In some implementations, the peripheral device 410 is a prosthesis, an exoskeleton, an orthotic, and/or an exosuit. In some implementations, the prosthesis is, but is not limited to, a robotic limb prosthesis (e.g., a robotic arm or leg prosthesis), a robotic hand prosthesis, and/or a robotic foot prosthesis. In some implementations, the exoskeleton is, but is not limited to, a hip exoskeleton, a knee exoskeleton, an ankle exoskeleton, and/or a multiple joint exoskeleton. In some implementations, the orthotic is, but is not limited to, a robotic foot orthotic, a robotic leg orthotic, a robotic ankle orthotic, a robotic knee brace, a robotic arm brace, a robotic leg brace. In some implementations, the exosuit is, but is not limited to, a soft wearable robot composed of a textile. In some implementations, the exosuit excludes an external rigid structure.

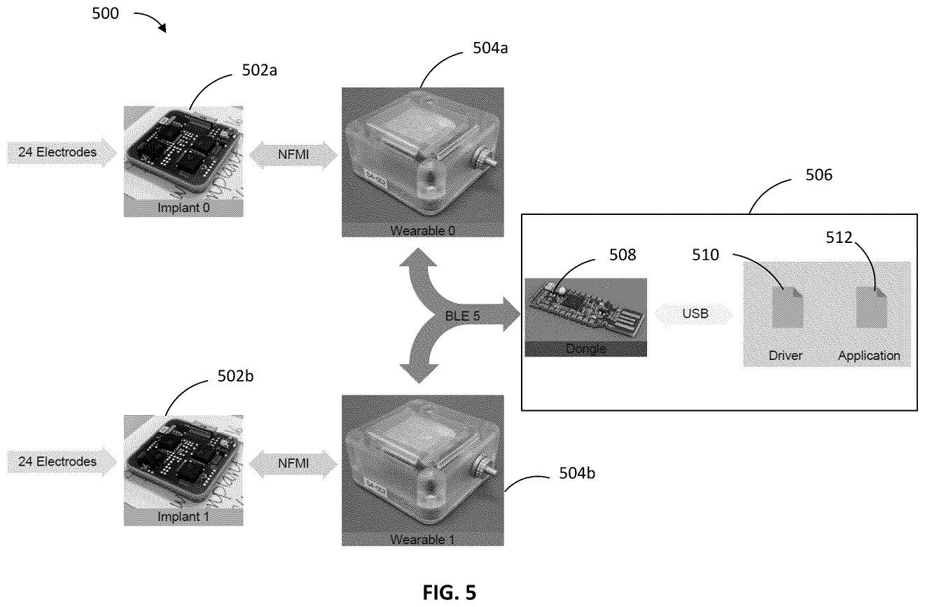

FIG. 5 is a block diagram of an example implantable system 500. The implantable system includes two implants 502a-b, two wearable devices 504a-b, and an EPU 506. The implants 502a-b include 24 electrodes each to measure EMG signals from skeletal muscles. The implants 502a-b communicate with the wearable devices 504a-b via an NFMI communications link. The wearable devices 504a-b communicate with the EPU 506 via a short range wireless communications link (e.g., a Bluetooth® Low Energy communications link). The EPU 506 includes a communications dongle 508 connected via USB to a driver 510, and an application 512.

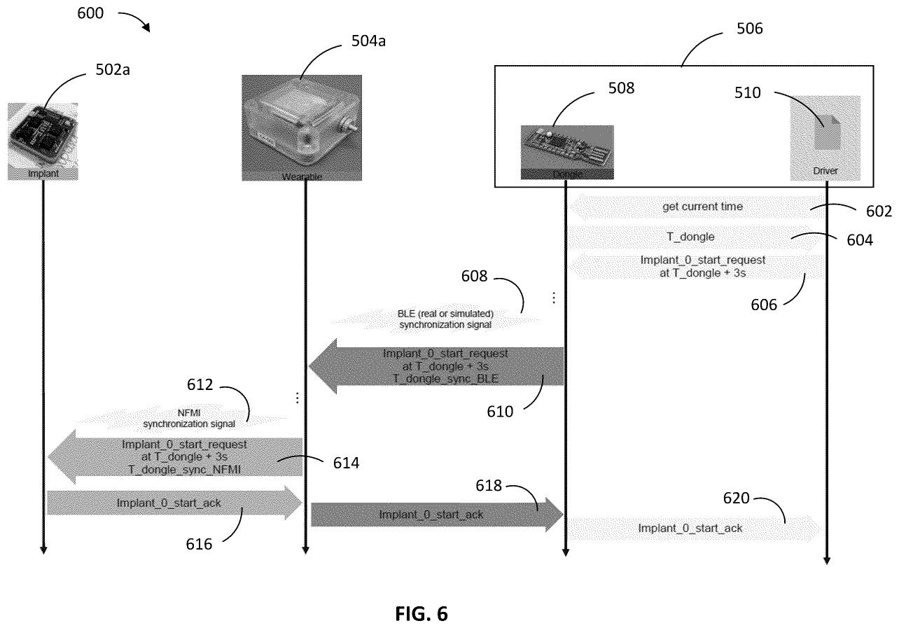

FIG. 6 is a timing diagram of an example process 600 for synchronizing the implantable system 500. The driver 510 sends a request to the communications dongle 508 to get the current time of the dongle (602). The communications dongle 508 returns its current clock time to the driver 510 (604). The driver 510 sends a request to the communications dongle 508 to start taking data from the implant 502a (606). The request includes a data acquisition start time of the communications dongle time (T_dongle) plus a time delay of 3 seconds.

The communications dongle 508 sends a synchronization signal to the wearable device 504a (608) via a short range wireless communications link. In some implementations, the synchronization signal is simulated if the communications dongle 508 does not receive a synchronization trigger in an expected location based on the synchronization period of the communications dongle 508. The communications dongle 508 forwards the implant start request including the data acquisition start time to the wearable device 504a (610). The wearable device 504a synchronizes its time to the clock time of the communications dongle 508 by applying steps 202-208 of the method 200. For example, the wearable device 504a determines a time offset based on the time of receipt of the synchronization signal and the time forwarded by the communications dongle 508 (T_dongle_sync_BLE).

The wearable device 504a transmits a synchronization signal to the implant 502a via an NFMI communications link (612). The wearable device 504a forwards the implant start request including the data acquisition start time and the time of transmittal of the NFMI sync signal in the dongle clock reference (T_dongle_sync_NFMI) (614). The implant synchronizes its clock, for example, by applying the method 250.

The implant 502a transmits a start acknowledgement to the wearable device 504a (616). The wearable device 504a forwards the start acknowledgement to the communications dongle 508 (618). The communications dongle 508 (620) forwards the start acknowledgement to the driver 510 (620). The implant 504a starts transmitting data to the wearable device 504a at the designated data acquisition start time. The wearable device 504a forwards the transmitted data to the communications dongle 508.

The wearable device 504b and the implant 502b can be synchronized following the same process 600. The EPU 506 can synchronize first with the wearable device 504a and the implant 502a several times (e.g., 5 times). The EPU 506 can then synchronize with the wearable device 504b and the implant 502b several times (e.g., 5 times). The number of synchronization iterations can be based on an allowable synchronization error. The time delay determined by the driver 510 for the data acquisition start time can be long enough to allow all wearable devices and implants communicatively connected to the EPU 506 to be synchronized the desired number of iterations. For example, if more devices are connected to the EPU 506 or if more synchronization iterations are desired, the time delay can be increased.

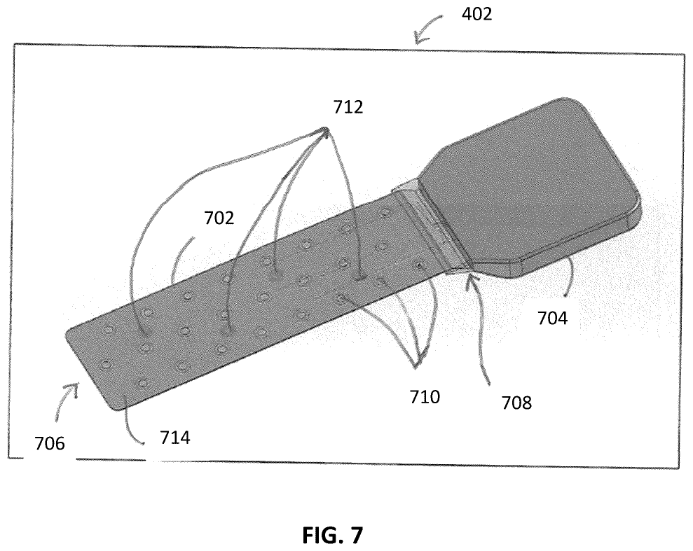

Referring to FIG. 7, the implantable device 402 is configured to be implanted in a subject, on the surface of one or more muscles of the subject. In some implementations, the muscle is a skeletal muscle or a portion thereof. In some implementations, the implantable device 402 is implanted in the subject such that the implantable device 402 is in direct contact with at least a portion of a fascia of the muscle, an epimysium of the muscle, a perimysium of the muscle, an endomysium of the muscle, a fascicle of the muscle, a muscle fiber, a tendon, a blood vessel of the muscle, a nerve of the muscle, or any combination thereof. In some implementations, the fascia is a deep fascia of the muscle. In some implementations, the deep fascia is an aponeurotic fascia and/or an epimysial fascia. In some implementations, the implantable device 402 is implanted in the subject such that the implantable device 402 is in direct contact with at least a portion of loose connective tissue of the muscle. In some implementations, the implantable device 402 is implanted in the subject such that the implantable device 402 is in direct contact with at least a portion of a surface of a fasciculus of the muscle.

In some implementations, the implantable device 402 can be inserted under the skin through one or more small incisions (e.g., an incision having a length of about 0.5 centimeters (cm) to about 5 cm). For example, a small flexible camera can be placed at the tip of an insertion tool to provide the surgeon with a clear view of where the insertion tool is located in space to ensure accuracy and safety during pocket formation through a limited number of (e.g., one or more) incisions. Once the proper implant pocket length is achieved, the implantable device 402 can then be inserted into the implant pocket and deployed onto the surface of one or more muscles. In some implementations, the implantable device 402 is not fixedly secured to the muscle. In some implementations, at least a portion of the implantable device 402 can be secured in place via one or more sutures, surgical glues, or physical anchoring features of the implantable device 402 used to fix the implantable device 402 to the underlying or overlying tissues. In some implementations, the implantable device 402 is configured to be sterilized (e.g., via autoclaving, gas sterilization, gamma radiation, etc.) prior to implantation.

The implantable device 402 includes an implantable substrate 702 and an electronic module 704 that are configured to operatively connect to each other, for example via a connector and/or a feedthrough architecture. The implantable substrate 702 is an elongated, generally flat substrate or strip having a proximal end 706 and a distal end 708. The implantable substrate 702 (e.g., an implantable sensor array substrate) includes one or more sensors (e.g., EMG sensors) 710, one or more reference electrodes 712, and an interconnect to electrically bond the one or more sensor pads 710 (e.g., EMG sensors or EMG electrodes) at the distal end 708. In some implementations, the implantable substrate does not include one or more reference electrodes and/or biasing electrodes. In some implementations, the reference electrodes 712 are biasing electrodes. In some implementations, the implantable substrate 702 includes one or more reference electrodes and one or more biasing electrodes. In some implementations, the sensors 710 are sensor pads. The electronic module 704 includes an opposing, second mating connector (e.g., a male or female connector) or feature configured to connect to the mating portion of the first connector of the implantable substrate 702. The electronic module 704 can include a case that houses the electronic components. In some implementations, the electronic module may not include a case that houses the electronic components. Instead, the electronic module can include a protective coating using technologies such as Atomic Layer Deposition (ALD) or Parylene C coating.

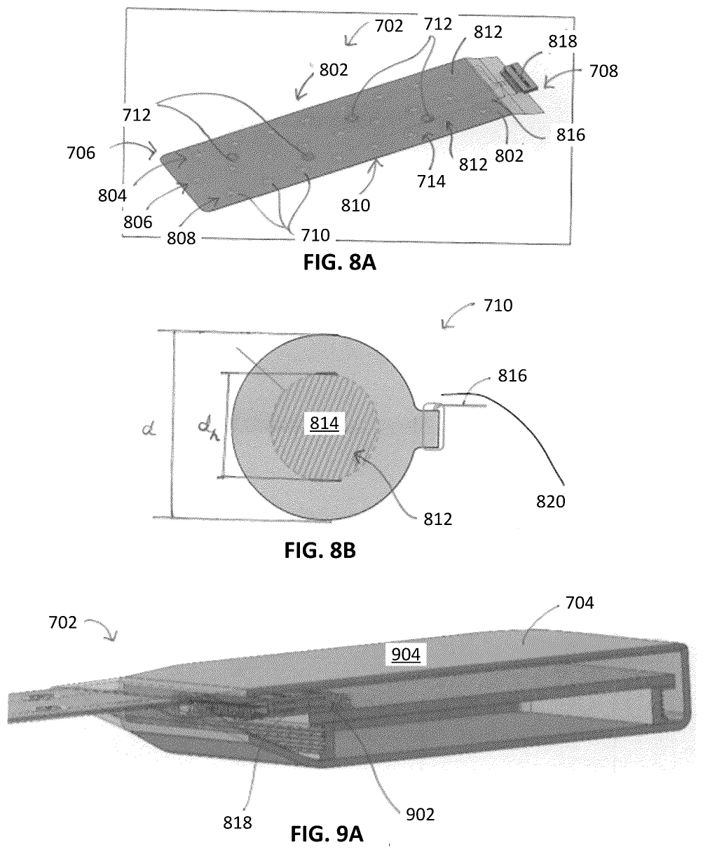

Referring to FIG. 8A, the implantable substrate 702 includes an electrode array 802 having three rows and eight columns of sensors 710 arranged in a grid configuration, for a total of twenty four sensors 710. The first row 804 and the third row 808 of sensors 710 are laterally aligned while the second row 806 of sensors 710 is longitudinally offset from the first and third rows 804, 808. The electrode array 802 further includes a pair of reference electrodes 712 that are staggered between the first and second rows 804, 806 and a pair of reference electrodes 712 that are staggered between the second and third rows 806, 808, for a total of four reference electrodes 712. The reference electrodes 712 are configured to be used as reference and bias drive. In some implementations, the electrode connections can be reconfigured in situ. For example, in some implementations, this can be implemented using analogue switches and/or multiplexers, which are controlled by a microcontroller. In some implementations, the degree of reconfigurability depends on which exact components with suitable parameters can be sourced. For example, in some implementations, fewer electrode configuration options can be implemented with single-pole, double-throw switches compared to a full switch matrix.

The center of each sensor 710 is about equidistant from the center of each of the neighboring sensors 710. In some implementations, two or more sensors 710 are spaced equidistantly from a center of each of the two or more sensors 710. In some implementations, the center-to-center sensor 710 spacing is about 10 mm. In some implementations, the distance between the center of each sensor 710 and the center of an immediately adjacent sensor 710 is about 10 mm.

Alternative numbers of columns and rows may be employed. For example, in some implementations, 4 or more electrodes are distributed into multiple rows and multiple columns. Also, every row need not contain the same number of columns. For example, an implantable substrate can include a design having one or more rows that include 10 columns of electrodes while additional rows can include 4 or more rows of electrodes to enable a greater amount of electrical field resolution.

The sensors 710 are biocompatible, electroconductive electrodes that are configured to contact a surface of a muscle in a subcutaneous, subadipose, or subfascial area of the subject and are configured to measure an electrical biopotential of the muscle. In some implementations, the sensors 710 are EMG sensors. In some implementations, the electrode array 802 includes about 4 to about 30 sensors 710. In some implementations, the electrode array 802 includes about 35 to about 50 sensors 710. In some implementations, the sensors 710 are platinum-iridium alloy electrodes. In some implementations, the sensors 710 are carbon-based electrodes. In some implementations, the sensors 710 are any other suitable type of biocompatible and/or bioinert metal such as titanium, or a biocompatible and/or bioinert polymer such as PEDOT (poly 3,4-ethylenedioxythiophene). In some implementations, the reference electrodes 712 are platinum iridium electrodes. In some implementations, the reference electrodes 712 are carbon-based electrodes. In some implementations, the reference electrodes 712 are any other suitable type of biocompatible and/or bioinert metal such as titanium, or a biocompatible and/or bioinert polymer such as PEDOT (poly 3,4-ethylenedioxythiophene). In some implementations, the sensors 710 are configured to have an impedance ranging from about 0.4 kiloOhm (kOhm) to about 1 MOhm (e.g., about 0.4 kOhm to about 0.5 kOhm, about 0.4 kOhm to about 0.6 kOhm, about 0.4 kOhm to about 0.7 kOhm, about 0.4 kOhm to about 0.8 kOhm, about 0.4 kOhm to about 0.9 kOhm, or about 0.7 kOhm to about 1 kOhm, about 1 kOhm to about 100 kOhm, about 100 kOhm to about 250 kOhm, about 100 kOhm to about 500 kOhm, about 100 kOhm to about 1 MOhm, about 500 kOhm to about 1 MOhm, about 1 kOhm to about 1 MOhm, or about 100 kOhm to about 500 kOhm) at 1 kHz.

The sensors 710 and reference electrodes 712 along with their wires 816 are embedded within the implantable substrate 702. The implantable substrate 702 is composed of a flexible and bioinert and/or biocompatible material. In some implementations, the implantable substrate 702 is composed of silicone. Non-limiting examples of materials that the implantable substrate can be composed of include polymer-based materials (such as but not limited to silicone, liquid crystal polymer, or shape memory polymer) and a thin-film substrate coated with one or more biocompatible insulators (such as but not limited to silicone-carbide, silicone-oxide, or silicone-nitride). In some implementations, the implantable substrate is configured to wrap around a muscle. In some implementations, the implantable substrate is configured to wrap around a tissue having a generally cylindrical or tubular structure (e.g., a muscle of a limb). In some implementations, the implantable substrate is configured to wrap around a circumference of one or more muscles of the subject when implanted.

The implantable substrate 702 has a top surface 714 and a bottom surface 810 opposing the top surface 714. The top surface 714 includes the sensors 710, and the bottom surface 810 includes the reference electrodes 712. In some implementations, the sensors 710 are embedded within the top surface 714, and the reference electrodes 712 are embedded within the bottom surface 810. The top surface 714 is configured to be in contact with the muscle of the subject and defines one or more holes 812 to expose the sensors 710, thereby facilitating sensor 710-to-muscle contact.

Referring to FIG. 8B, the sensors 710 have circular shape that is concentric with the holes 812. The sensors 710 have a diameter d of about 4 millimeters (mm), and the holes 812 have a diameter dh of about 2 mm. In some implementations, the sensors have a diameter d that is larger than the diameter dh of the holes 812. In some implementations, the diameter of the sensors 710 is about 50% to about 60% (e.g., about 50% to about 55% or about 55% to about 60%) larger than the diameter of the holes 812. The sensor 710 has an exposed area 814 that is configured to contact a muscle of the subject and is about 50% to about 60% larger than the area of the sensor 710. In some implementations, the exposed area 814 can include a visual marker (e.g., a number or letter) that identifies one or more of the sensors 710 and the reference electrodes. In some implementations, the top and bottom surfaces 714, 810 include a visual marker (e.g., a number or letter) or are colored differently to be distinguished from each other.

The wire 816 of each sensor 710 and reference electrode 712 is laser welded to the surface of its corresponding sensor 710 or reference electrode 712 at a laser weld joint 820. The sensor 710 or reference electrode 712 and the laser welded interface is encapsulated in a bioinert and/or a biocompatible material (e.g., silicone) to protect the electrical connection from the environment. The wire 816 can be composed of but is not limited to a conductive polymer, metal alloy, or carbon-based material.

Referring again to FIG. 8A, the implantable substrate 702 typically has a length (e.g., in a direction extending from the proximal end 706 of the implantable substrate 702 to the distal end 708 of the implantable substrate 702) of about 10 mm to about 300 mm and a width (e.g., extending across the lateral edges of the implantable substrate 702 of about 10 mm to about 200 mm. The implantable substrate 702 typically has a total thickness of about 0.5 mm to about 5 mm, providing the implantable device 402 with a film-like substrate having increased flexibility, which may be less noticeable to the subject when the implantable device 402 is implanted. The implantable substrate 702 has a generally rectangular shape with rounded edges; however, the implantable substrate can have any other suitable shape. In some implementations, the implantable substrate 702 is sized to be wrapped around one or more muscles of a subject at a subcutaneous, subadipose, or subfascial depth.

The implantable device further includes a connector 818 at the distal end 708 of the implantable substrate 702. In some implementations, the connector 818 is a male connector. In some implementations, the connector 818 is a female connector. In some implementations, the connector 818 is a pin connector. In some implementations, the connector 818 is a pigtail or mating unit designed to feed into and join with a receiving unit via hermetically enclosed physical contact. In some implementations, the connector 818 is bare wire 816. The wires 816 leading from each of the sensors 710 and reference electrodes 712 are affixed to the connector 818 via laser welding (or some alternative means of bonding), thereby fixedly securing the sensors 710 and the reference electrodes 712 to the connector 818. When the connector 818 is composed of bare wire 816, the wire 816 is laser welded or bonded in some fashion directly to the connector 902. In some implementations, the connector 818 is a high-density connector. For example, the connector 818 can have many individual contacts (e.g., a contact for each electrode) within a relatively small space.

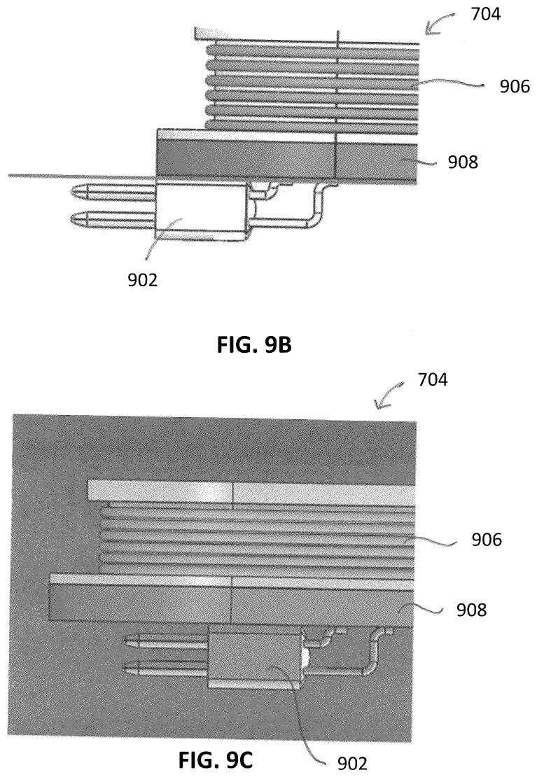

Referring to FIGS. 9A-9C, the implantable substrate 702 includes an electronic module 704 including a connector 902 configured to connect to connector 818, thereby connecting the electronic module 704 to the implantable substrate 702. In some implementations, the connector 902 is a female connector. In some implementations, the connector 902 is a male connector. In some implementations, the connector 902 is a socket connector. In some implementations, the connect 902 is a pin connector. In some implementations, the connector 902 is a feedthrough connector.

The electronic module 704 further has a case 904 defining an enclosed space that houses one or more components (e.g., electronic components, a magnet, a sensor, and/or the like) of the implantable device. In some implementations, the case 904 is composed of or coated with a biocompatible material and/or a bioinert material. In some implementations, the case 904 is a hermetic enclosure that prevents fluid ingress and egress. In some implementations, the case 904 is a rigid structure that provides physical protection for the components within it. In some implementations, the case 904 is composed of a thermoplastic polymer (e.g., polyether ether ketone (PEEK)).

The components disposed within the open, interior space of the case 904 include a power receiver coil 906 configured to facilitate wireless inductive charging, wireless power transfer, and/or wireless communication of the implantable device, a printed circuit board (PCB) 908 including electronic components configured to acquire, process, and/or transmit the sensor signals, a capacitor configured to store a minimal amount of charge or power to survive short power losses on the order of seconds, and a motion sensor configured to capture, measure, and/or transmit motion data of the implantable device. In some implementations, the PCB 908 contains other electronic components such as, but not limited to, an optical sensor (e.g., a photoplethysmography (PPG) sensor, a peripheral oxygen saturation (SpO2) sensor, or the like), a pressure sensor, a force sensor, a humidity sensor, a temperature sensor, a chemical sensor, a location sensor, and/or a positioning sensor. In some implementations, the motion sensor is an inertial measurement unit (IMU). In some implementations, the motion sensor is a micro-electro-mechanical-system (MEMS)-based IMU. In some implementations, the motion sensor is a combined accelerometer and gyroscope. The electronic module 704 does not include a battery or a Bluetooth® wireless communication component given that the wearable device provides these features.

Referring specifically to FIGS. 9B and 9C, in some examples, the connector 902 can be secured to a surface of the PCB 908 at various positions. For example, FIG. 9B illustrates the connector 902 being surface-mounted to the PCB 908 and being flush from the edge of the PCB 908 to the face of the connector 902. In another example, FIG. 9C illustrates the connector 902 being surface-mounted to the PCB 908 and being offset from the edge of the PCB 908. In some implementations, the connector 902 is contained within the wall structure of the case 904. In some implementations, the power receiver coil 906 and electronic components within the electronic module 704 are coated with parylene to waterproof these components and add dry lubricity. In some implementations, the electronic module 704 includes two separate coils for wireless power (e.g., the power receiver coil 906) and wireless communications. The power receiver coil 906 is wound on a bobbin with the same outline as the PCB 908 and sits directly on it. In some implementations, the power receiver coil 906 is embedded within the PCB 908 itself. In some implementations, the power receiver coil 906 is embedded within the housing 1102. The communications coil is a smaller solenoid-style coil mounted on a location inward on the PCB 908.

The implantable device uses a Near Field Magnetic Induction (NFMI) link to communicate with the wearable device. Sensor data (e.g., EMG data and/or motion data) is configured to be primarily sent from the implantable device to the wearable device over this link. Command signals and control signals are also configured to be transmitted over this link; for example, the supply voltage and current of the implantable device can be transmitted to the wearable device, and the wearable device can update settings for the wireless power transmitter over this link. Additionally, the wearable device is configured to transmit data to the implantable device over the NFMI link. In some implementations, data is transferred directly over the power link (via a radiofrequency modulation scheme). In some implementations, communication between the wearable device and the implantable device is accomplished via other suitable methods including, but not limited to, methods using galvanic, capacitive, ultrasound, optical, and molecular components.

In some implementations, the implantable device is powered over a wireless power system using a magnetic link. In some implementations, there is no significant energy storage on the implantable device; thus, the wireless link is configured to be on constantly while the system is in use. In some implementations, the output voltage of the power receiver coil 906 is rectified and smoothed, resulting in an unregulated voltage from which all other power supplies are generated.

In some implementations, the electronics module further includes an integrated current, voltage, and power measurement circuit configured to measure the voltage received by the implantable device and the current drawn by it. In some implementations, measuring the voltage received and the current drawn enables an alignment assistance function of the wearable device and closed-loop power control, if necessary.

In some implementations, the electronic module further includes a microcontroller (MCU) configured to capture data from an analogue front-end and forward it to the NFMI chip, along with system configuration and monitoring functions. In some implementations, the MCU is a part of the NFMI chip. In some implementations, the MCU is a component that is separate from the NFMI chip.

In some implementations, the electronic module further includes an analog front end in order to perform analog signal processing such as filtering, noise reduction, and/or digitization of the signals.

In some implementations, the electronics module further includes anti-aliasing circuits and/or buffers, multiplexers, and averaging circuits. In some implementations, the electronics module may include additional components for digital signal processing.

An implantable system may be substantially similar in construction and function in several aspects to the implantable systems 400 discussed above but can include an alternative implantable device 1000 instead of the implantable device 402. In some implementations, the implantable device 1000 may have different connectors and an electronic module having a rigid, hermetic case composed of ceramic. For example, the implantable device 1000 may have a hermetic feedthrough connector. Such hermetic connectors and hermetic case can prevent ingress and egress of fluids when implanted in the body, can provide an electronic module with a slimmer profile, and can act as a protective casing for impact resistance. The implantable device 1000 is respectively part of the implantable system 400 that otherwise includes a wearable device 404 that can be removably secured to a limb of the subject 406, an external processing unit (EPU) 408, and a peripheral device 410.

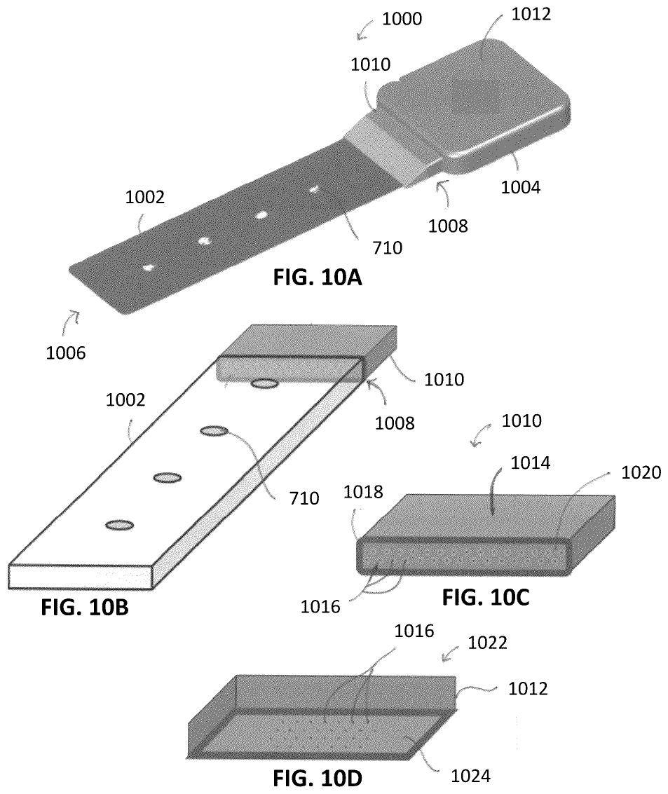

Referring to FIGS. 10A-10D, the implantable device 1000 includes an implantable substrate 1002 and an electronic module 1004 that are configured to operatively connect to each other through a connector. The electronic module 1004 includes a case 1012 that houses the electronic components. The case 1012 is composed of a ceramic material. The implantable substrate 1002 is an elongated, generally flat substrate or strip having a proximal end 1006 and a distal end 1008. The implantable substrate 1002 (e.g., an implantable sensor array substrate) includes one or more sensors (e.g., sensor pads or EMG sensors) 710, and one or more reference electrodes. The electronic module 1004 includes a feedthrough connector 1010 (e.g., a metal feedthrough connector) including a plurality of feedthroughs 1016 defined by a side surface 1020 and configured to directly connect to the wires of the sensors 710 and to the reference electrodes 712 of the implantable substrate 1002 at the distal end 1008. In some implementations, the feedthrough connector 1010 is a hermetic electrical contact feedthrough connector whereby the conductive leads of the array 702 converge into one or more pigtails that connect to the electronic module 1004. In some implementations, the feedthrough connector 1010 is a metal feedthrough connector. The feedthrough connector 1010 includes a case 1014 with generally orthogonal dimensions including a metal flange 1018 framing the side surface 1020 for welding of a multi-part hermetic enclosure. The case 1014 can be composed of a biocompatible material such as but not limited to ceramic, metal, thermoplastic, and/or any other rigid or semi-rigid polymer.

Referring specifically to FIG. 10D, an alternative feedthrough connector 1022 includes a plurality of feedthroughs 1016 defined by a bottom surface 1024 instead of a side surface, as in the feedthrough connector 1010 of FIGS. 10B and 10C.

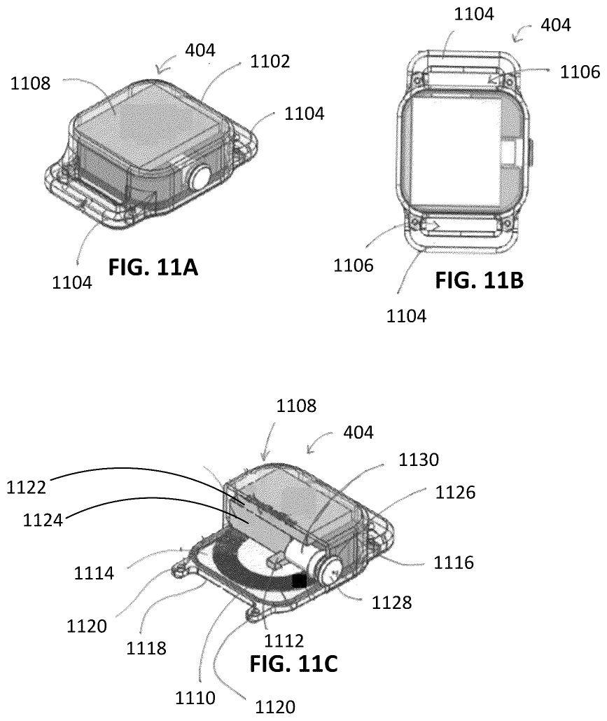

Referring to FIGS. 11A-11C, the wearable device 404 is an external module that is configured to communicate with and power the implantable device. The wearable device 404 is configured to send power to the implantable device and is configured to serve as a bridge between the implantable device and an external processing unit (e.g., a smartphone, a computer, a prosthesis, etc.).

The wearable device 404 has a generally square shape; however, the wearable device can have any suitable shape (e.g., a low profile disc or a low profile square), dimensions, and/or configuration. The wearable device 404 has a housing 1102 defining an interior space configured to house one or more components (e.g., electronic components). The housing 1102 includes a cover 1116 that is configured to mate and be securely fixed to a base 1118, thereby forming the enclosed space that houses the components. The cover 1116 and base 1118 are configured to be securely fixed to each other by a pair of retainers 1120 (e.g., bolts). In some implementations, other suitable methods of securely fixing the cover 1116 to the base 1118 (e.g., via a snap fit connection, an adhesive, a glue, etc.) can be used. To further secure the connection between the cover 1116 and the base 1118, the main body 1108 includes a sealing member disposed around the four edges of the main body 1108. The sealing member is configured to provide a water-resistant seal formed between the cover 1116 and the base 1118 when the cover 1116 and the base 1118 are coupled to form the interior space housing the components.

The housing 1102 includes a pair of lugs 1104. Each lug 1104 is symmetrically arranged on opposing sides of the main body 1108 of the housing 1102. Each lug 1104 is integrally connected to the main body 1108 and extends outwardly from opposing edges of the main body 1108. Each lug 1104 defines a slot 1106 configured to receive a strap that can be used to attach the wearable device 404 to a subject, for example. The components disposed within the interior space of the housing 1102 include, for example, a power transmitter coil 1110 configured to power the implantable device over the wireless link via an inductive magnetic field, a communication coil 1112 configured to facilitate wireless communication, and a battery 1122 that is self-contained and configured to supply power to the electronic components of the wearable device 404. In some implementations, the housing 1102 does not contain lugs 1104 and slots 1106, but rather contains structural features designed to snap on, slide in, or affix a strap that can be used to attach the wearable device 404 to a subject.

The power transmitter coil 1110 is configured to sit on an internal surface 1114 of the base 1118, within the internal space defined by the main body 1108 of the housing 1102. As described above, the wearable device 404 is configured to power the implantable device over a wireless link. The power transmitter coil 1110 includes an amplifier to drive the coil that will generate a magnetic field. In some implementations, the magnetic link of the power transmitter coil 1110 is configured to use an operating frequency that is greater than an operating frequency to be used by the NFMI communications link to increase separation and prevent undesirable electromagnetic interference. In some implementations, the amplifier is a high efficiency amplifier. In some implementations, the amplifier is configured to keep the end-to-end efficiency of the wireless power link as high as possible, thereby extending the battery life as much as possible. In some implementations, the power transmitter coil 1110 is configured to be controllable to implement a closed loop control of the wireless power link, if required.

At least a portion of the communication coil 1112 is disposed on an internal surface 1114 of the base 1118, within the internal space defined by the main body 1108 of the housing 1102, and in close proximity to the power transmitter coil 1110, as shown in FIG. 11C. The configuration and construction of the communication coil 1112 is similar to the communication coil in the implantable device.

The battery 1122 is disposed over the PCB 1124 within the internal space defined by the main body 1108 of the housing 1102. The battery 1122 is a rechargeable battery configured to be charged when an external power source is connected to it. In some implementations, the battery 1122 is a lithium-ion battery. In some implementations, the battery 1122 is a pouch cell battery with built-in protection circuitry. In some implementations, the battery 1122 is a prismatic cell with built-in protection circuitry. In some implementations, the battery 1122 is a lithium-ion pouch cell or prismatic cell battery with built-in protection circuitry. In some implementations, the battery 1122 has a battery capacity configured to support a 2-hour data acquisition time and an additional hour for preparation and alignment. In some implementations, the battery 1122 is configured to support about 2 hours (h) to about 24 hours (e.g., about 2 h to about 3 h, about 2 h to about 4 h, about 2 h to about 5 h, about 2 h to about 6 h, about 2 h to about 7 h, about 2 h to about 8 h, about 2 h to about 9 h, about 2 h to about 10 h, about 2 h to about 11 h, about 2 h to about 12 h, about 2 h to about 14 h, about 2 h to about 16 h, about 2 h to about 18 h, about 2 h to about 20 h, about 2 h to about 24 h, about 12 h to about 24 h) of data acquisition time. In some implementations, the battery 1122 is sized to fit within the enclosed space defined by the main body 1108. In some implementations, the battery 1122 is a cylindrical cell having a reduced surface area with respect to a pouch cell or a prismatic cell battery. In some implementations, the battery 1122 is a flexible and conformal substrate to accommodate unconventional form factors.

In some implementations, the wearable device 404 can be fully operational while simultaneously charging the battery 1122 when connected to an external power supply. In some implementations, this configuration is not foreseen to be necessary in a normal usage scenario, however, this configuration is configured to enable the run time of the wearable to be easily extended (e.g., by connecting it to an external power bank).

As described above, the wearable device 404 uses a Near Field Magnetic Induction (NFMI) link to communicate with the implantable device. Sensor data (e.g., EMG data and/or motion data) from the implantable device is configured to be received by the wearable device 404 over this link. Command signals and control signals are also configured to be transmitted over this link; for example, the supply voltage and current measurements of the implantable device for alignment and closed loop power control, if required. In some implementations, the wearable device 404 can update settings for the wireless power transmitter over this communication link.

In some implementations, the wearable device 404 communicates with an EPU (e.g., a personal computer (PC), a smartphone, or the like) via a short range communications link (e.g., a Bluetooth® link). In some implementations, the wearable device 404 includes an integrated Bluetooth® module or a Bluetooth® chipset to enable such communication. In some implementations, the wearable device 404 is configured to transmit sensor data (e.g., motion data and/or EMG data) to the EPU over the Bluetooth® link. In some implementations, the PCB 1124 includes a microcontroller configured to receive data (e.g., sensor data) sent from the implantable device over the NFMI link and is configured to forward the data to an EPU (e.g., PC, smartphone, or the like) via the Bluetooth® link. In some implementations, the microcontroller is configured to forward system configurations and monitoring functions to the EPU via the Bluetooth® link. In some implementations, the PCB 1124 includes a decoder configured to decode the EMG signals on the wearable device 404.

As shown in FIGS. 11A-11C, the wearable device 404 includes a connector 1126 configured to allow access to an external device (e.g., an external power supply and/or an EPU). Thus, the connector 1126 is configured to enable charging of the battery 1122 as well as configuration and debugging of the assembled wearable device 404 in the field. The connector 1126 includes a connector cap 1128 and a shaft 1130. The connector cap 1128 is external to the housing 1102 and is removably coupled to the shaft 1130. The shaft 1130 extends through the housing 1102, within the enclosed area defined by the main body 1108. As the connector 1126 is a major potential ingress point in an otherwise sealed device in a harsh environment, the connector 1126 provides an appropriate level of ingress protection and robustness. In some implementations, the connector cap 1128 can be a blanking cap, a plug, or a push-pull connector. In some implementations, the connection between the connector cap 1128 and the shaft 1130 is a watertight connection and/or a vacuum tight connection. In some implementations, the connector 1126 is configured to support signal transmission (e.g., ground signals, power transmission, debugging signals, or the like) via a USB having a modified terminal configured to couple with the connector 1126. In some implementations, the connector 1126 includes about 4 pins to about 12 pins.

The wearable device 404 may have a variety of ways of providing feedback to the user about particular conditions (e.g., if there is an active alignment assistance or a need to communicate a state such as, but not limited to, Bluetooth® pairing, confirmation of power on and/or off. In some implementations, the feedback is a direct visual feedback, where the wearable device 404 incorporates an indicator light (e.g., a light emitting diode (LED) along with a light pipe/guide) disposed on the outside of the housing 1102. In some implementations, the indicator light is disposed within the internal space defined by the main body 1108 of the housing 1102. In some implementations, the wearable device 404 provides tactile feedback, where the wearable device 404 can vibrate, buzz, or otherwise stimulate the user's sense of touch. In some implementations, the wearable device 404 provides auditory feedback, where the wearable device 404 can beep, click, or otherwise generate any other suitable type of sound. In some implementations, the wearable device 404 simultaneously provides visual, tactile, and auditory feedback.

In some implementations, the wearable device 404 includes a Hall effect switch configured to turn on in the presence of a magnet or magnetic field and turn off when the magnet or magnetic field is removed. For example, if it is necessary for the user to interact directly with the wearable device 404 (e.g., to wake it up from a low-power mode, initiate Bluetooth® pairing, or the like), a Hall effect switch can be configured to detect a magnet that is brought close to a defined location near the enclosure, defined by the main body 1108, where the Hall effect switch is located. In some implementations, using a Hall effect switch instead of a physical switch advantageously keeps the user interaction a contactless one, where the enclosure can remain completely sealed, without risking the creation of an ingress path via a switch.

In some implementations, a capacitive sensor can also be used to switch between modes in place of the Hall effect switch. In some implementations, the capacitive sensor is configured to measure the change in capacitance when the user's finger is brought near the capacitive sensor. Like the Hall effect sensor, this capacitive sensor makes the user interaction contactless, where the enclosure can remain completely sealed, thereby improving fluid ingress protection.

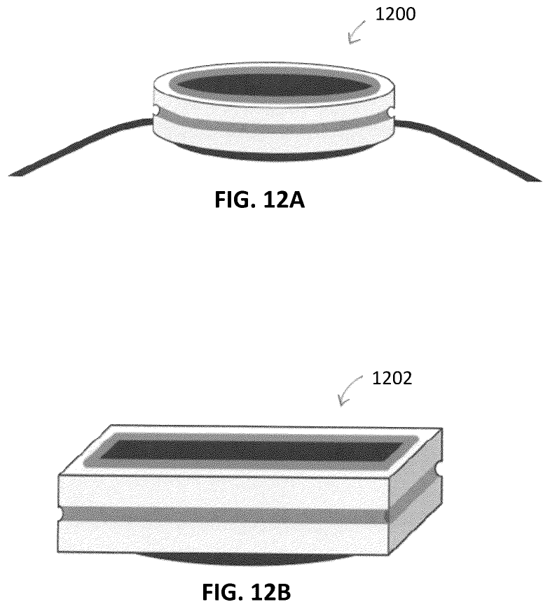

Referring to FIGS. 12A-B, an implantable system may be substantially similar in construction and function in several aspects to the implantable systems 400 discussed above but can include a first wearable device 1200 or a second wearable device 1202 instead of the wearable device 404. In some implementations, the first or second wearable devices 1200, 1202 may have a different shape and/or reduced dimensions with respect to the wearable device 404 shown in FIGS. 11A-C. For example, the first wearable device 1200 has a circular shape and the second alternative wearable device 1202 has a rectangular shape. Such exemplary shapes and reduced dimensions can provide a wearable device with a slimmer profile, which can enhance the form factor and comfort of the wearable device 104. The first wearable device 1200 or a second wearable device 1202 is respectively part of the implantable system 100 that otherwise includes an implantable substrate 102 (or an implantable substrate 1000), an external processing unit (EPU) 108, and a peripheral device 110.

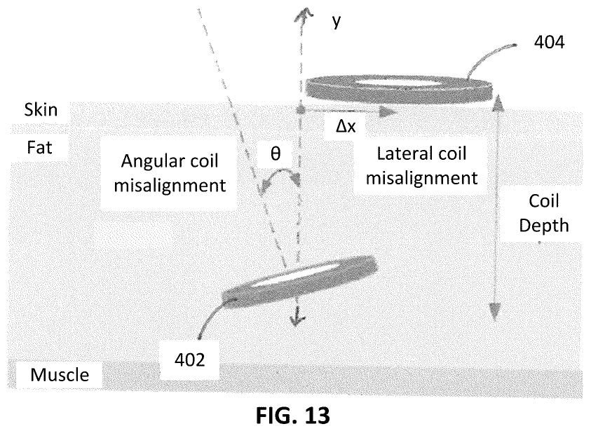

FIG. 13 illustrates examples of angular coil misalignment, lateral coil misalignment, and depth between the wearable device 404 on the skin surface and an implanted implantable device 402 in close proximity to a muscle surface. The implantable device 402-to-wearable device 404 interface is between the electronic components and magnet of the implantable device 402 to the external bridging hardware of the wearable device on the skin surface. This interface is important to ensure reliable data collection and transmission between the implantable device 402 and the wearable device 404. The interface also includes a power link between the power receiver coil and the power transmitter coil that is critical to power up the implantable device 402.

In some implementations, the lateral coil misalignment can be defined as the distance x between the center of the power receiver coil of the implantable device 402 and the rim or an edge of the power transmitter coil of the wearable device 404. In some implementations, the implantable device 402 and wearable device 404 can have a lateral coil misalignment of about 5 mm to about 15 mm at most (e.g., about 5 mm to about 6 mm, about 5 mm to about 7 mm, about 5 mm to about 8 mm, about 5 mm to about 9 mm, about 5 mm to about 10 mm, about 5 mm to about 11 mm, about 5 mm to about 12 mm, about 5 mm to about 13 mm, about 5 mm to about 14 mm, about 5 mm to about 10 mm, or about 10 mm to about 15 mm) for ideal functioning of the implantable device 402 and the wearable device 404 (e.g., having reliable data transmission and collection and powering up of the implantable device 102).

In some implementations, the angular coil misalignment can be defined as the angle theta (0) of the implantable device 402 relative to the Y-axis, which extends through the center of the power receiver coil of the implantable device 402 and is adjacent to the rim or an edge of the power transmitter coil of the wearable device 404. In some implementations, the implantable device 402 and wearable device 404 can have an angular coil misalignment of about 5 degrees to about 15 degrees at most (e.g., about 5 degrees to about 6 degrees, about 5 degrees to about 7 degrees, about 5 degrees to about 8 degrees, about 5 degrees to about 9 degrees, about 5 degrees to about 10 degrees, about 5 degrees to about 11 degrees, about 5 degrees to about 12 degrees, about 5 degrees to about 13 degrees, about 5 degrees to about 14 degrees, about 5 degrees to about 15 degrees, about 5 degrees to about 10 degrees, or about 10 degrees to about 15 degrees) in any direction for ideal functioning of the implantable device 102 and the wearable device 404 (e.g., having reliable data transmission and collection and powering up of the implantable device 402).

In some implementations, the coil depth can be defined as the subcutaneous depth of the implantable device 402, once implanted, relative to the skin surface and to the wearable device 404. In some implementations, the implantable device 402 can have a coil depth ranging of about 10 mm to about 50 mm at most (e.g., about 10 mm to about 11 mm, about 10 mm to about 12 mm, about 10 mm to about 13 mm, about 10 mm to about 14 mm, about 10 mm to about 15 mm, about 10 mm to about 16 mm, about 10 mm to about 17 mm, about 10 mm to about 18 mm, about 10 mm to about 19 mm, about 10 mm to about 20 mm, about 10 mm to about 21 mm, about 10 mm to about 22 mm, about 10 mm to about 23 mm, about 10 mm to about 24 mm, about 10 mm to about 25 mm, about 10 mm to about 26 mm, about 10 mm to about 27 mm, about 10 mm to about 28 mm, about 10 mm to about 29 mm, about 20 mm to about 30 mm, about 15 mm to about 30 mm, about 20 mm to about 40 mm, about 20 mm to about 50 mm, about 30 mm to about 40 mm, about 30 mm to about 50 mm, or about 40 mm to about 50 mm) for ideal functioning of the implantable device 402 and the wearable device 404 (e.g., having reliable data transmission and collection and powering up of the implantable device 402).

### Methods of Using the Implantable System

The implantable system of the disclosure can be used to control a peripheral device (e.g., a prosthesis or exoskeleton) and can be used to translate, interpret, or convert gestures or sign language into speech or words. For example, in some implementations, the methods of the disclosure include detecting sensor data (e.g., EMG sensor data and/or motion sensor data) from a subject using any of implantable devices disclosed herein. The method further includes wirelessly transmitting the sensor data from the implantable device to any of the wearable devices disclosed herein, wirelessly transmitting the sensor data from the wearable device to an external processing unit, processing the sensor data using one or more machine learning classifiers, and based on the processing step performed by the machine learning classifiers, generating one or more control outputs that lead to the translation, interpretation, conversion, and/or display of one or more gestures or sign language into an audible sound, speech, one or more words configured to be displayed on a screen (e.g., a screen of the EPU, a mobile device screen, a computing device screen, or the like), and/or one or more images configured to be displayed on the screen. In some implementations, the audible sound, speech, words, and/or images are reproduced, broadcast, and/or displayed in a same device. In some implementations, the audible sound, speech, words, and/or images are reproduced, broadcast, and/or displayed in one or more different and individual devices. In some implementations, the audible sound, speech, words, and/or images are simultaneously reproduced, broadcast, and/or displayed in a device. In some implementations, the device is operatively connected to the implantable system described herein.

Disclosed herein, in certain implementations, are methods of controlling a peripheral device (e.g., a prosthesis, an exoskeleton, and/or an exosuit). The methods include detecting sensor data (e.g., EMG sensor data and/or motion sensor data) from a subject using any of implantable devices disclosed herein. The method further includes wirelessly transmitting the sensor data from the implantable device to any of the wearable devices disclosed herein, wirelessly transmitting the sensor data from the wearable device to an external processing unit, processing the sensor data using one or more machine learning classifiers, and based on the processing step performed by the machine learning classifiers, generating a control output for the peripheral device (e.g., a prosthesis, an exoskeleton, and/or an exosuit).

As disclosed above, the data received by the EPU 408 from the wearable device 404 is configured to be processed by one or more algorithms. In some implementations, the algorithm is a machine learning classifier or machine learning model. In some implementations, the algorithm is trained with and is configured to classify either raw sensor data or sensor data with a pre-processing feature extraction. This sensor data includes but is not limited to EMG data and motion sensor data (e.g., IMU data).

In some implementations, the algorithm is a discrete classifier. In some implementations, the discrete classifier includes a determined number of predetermined output classes, each of which represents a different state for a peripheral device (e.g., a prosthesis, an exoskeleton, and/or an exosuit), such as a gesture, a joint angle, or a movement for a prosthesis. In some implementations, each of these output classes are mutually exclusive in their activation state, meaning that only one of the classes can be active at any time. In some implementations, the discrete classifier can be paired with a proportional control system, where the discrete classifier determines which degrees of freedom are moving, and a proportional signal (e.g., the integral of the absolute value of the EMG signal) determines the speed or torque of the degrees of freedom in motion. In some implementations, this discrete classifier can be an algorithm with a high number of hyperparameters, such as deep learning, or a low number of hyperparameters, like a logistic regression, linear discriminant analysis, or support vector machine classifier. In some implementations, other suitable types of algorithms that can be used to create this type of model.

In some implementations, the algorithm is a continuous classifier. In some implementations, the continuous classifier includes a determined number of outputs that can be simultaneously active. In some implementations, in the case of a control system for a prosthesis or exoskeleton, each output of the classifier controls a continuous value, such as, but not limited to, a joint angle, a torque, or an angular velocity of a single degree of freedom (DoF). In some implementations, other suitable types of algorithms that can be used to create this type of model.

In some implementations, the algorithm is trained before it processes and classifies sensor data. In some implementations, the training of the algorithm takes place on an EPU (e.g., on a smartphone, tablet, computing device, or the like) or on the cloud and required data input from the user. Once the implantable device has been subcutaneously implanted in the user, and the user is wearing the wearable device, the user can begin the training process. In some implementations, the training process starts by having the user connect her/his wearable device(s) to their EPU (e.g., a smartphone). In some implementations, the EPU includes an executable program (e.g., a mobile application) that is configured to facilitate the training process. Once the wearable device is connected to the EPU, the user can open a training menu on the executable program (e.g., a mobile application) to begin the training. A display (e.g., a screen) of the EPU is configured to display a virtual representation of the peripheral device (e.g., a prosthesis and/or an exoskeleton) of the user.

To train the algorithms based on the sensor data of the user, the virtual representation of the peripheral device (e.g., a prosthesis and/or an exoskeleton) on the display shows a series of movements that the user must perform with her/his body (e.g., the movement is performed with a residual limb when the peripheral device is a limb prosthesis) to the best of their ability. In some implementations, the implantable device is configured to capture sensor data (e.g., EMG signals and/or IMU signals) of the user and wirelessly transmit the sensor data to the wearable device. In some implementations, the wearable device is configured to wirelessly transmit the sensor data to the EPU or the cloud. When the user has provided all of the required data, the system trains the algorithms and generates the model parameters, which are loaded into the model stored locally on the EPU. These parameters determine how the input information is transformed into the desired outputs. The user can then use the entire system (e.g., implantable device, wearable device, and EPU, including the trained algorithm) to control a virtual peripheral device on an EPU interface to practice using their system. In some implementations, alternatively, the user can connect to a physical device (e.g., the prosthetic, the exoskeleton, an exosuit, or other peripheral device) and begin using their prosthetic, exoskeleton, or other peripheral device. For example, the EPU is then configured to transmit a control output to the prosthesis. In some implementations, the control outputs include, but are not limited to, a joint angle, a torque, a discrete gesture, an angular velocity of a single degree of freedom (DoF), one or more words, and one or more images. In some implementations, the output of a continuous model, for the control of a robotic device, may be one or more joint angles, voltage values, electric current values, and/or angular velocities. In some implementations, this continuous output, for some other peripheral device (e.g., a smartphone) may be a volume level, brightness level, and/or any other adjustable range of continuous values (e.g., settings on a smartphone). In some implementations, the output for a discrete model may be an integer, which corresponds to a gesture, word, phoneme, and/or image.

### ALTERNATIVE IMPLEMENTATIONS

While the above-discussed implantable devices and systems have been described and illustrated with respect to certain dimensions, shapes, arrangements, configurations, material formulations, and methods, in some implementations, an implantable device, that is otherwise substantially similar in construction and function to the implantable devices previously described herein, may include one or more dimensions, shapes, arrangements, configurations, and/or materials formulations that are different from the ones discussed above or may be used with respect to methods that are modified as compared to the methods described above.

For example, while the implantable device 402 has been described and illustrated as excluding a battery or a Bluetooth® wireless communication component given that the wearable device provides these features, in some implementations, an implantable device that is otherwise substantially similar in construction and function to the implantable device 402 may alternatively include an energy storage unit (e.g., a battery) such that the wearable device intermittently re-charges the implantable device. and/or a short-range communication link (e.g., a Bluetooth® wireless component), or some alternative component configured to enable wireless communications, to communicate directly with the external processing unit 408. For example, in some implementations, this configuration may also include a wearable device that is otherwise substantially similar in construction and function to the wearable device 404, that alternatively may exclude a an energy storage unit (e.g., a battery) and/or a short range communication link (e.g., a Bluetooth® wireless component), or some alternative component configured to enable wireless communications, given that one or more of these components may be provided in the alternative implantable device. In some implementations, in another example, the implantable system may not require a wearable device and may be a wearable-free, implantable system where the communications and power hardware are contained within and/or on the implantable device.

While the implantable systems and methods have been described and illustrated as including an EPU that is configured to wirelessly receive sensor data from the wearable device and is configured to wirelessly transmit a signal (e.g., a control output) to the peripheral device (e.g., a prosthesis, an exoskeleton, and/or an exosuit), in some implementations, an implantable system that is otherwise substantially similar in construction and function to the implantable systems previously described may exclude an EPU. For example, in some implementations, the wearable device is configured to wirelessly transmit the sensor data directly to the peripheral device, and the peripheral device is configured to process the sensor data and generate a control output in situ.

### OTHER IMPLEMENTATIONS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for wireless synchronization in an implantable system, the method comprising:
receiving, by a first computing device, a first synchronization signal;
recording, by the first computing device, a first timestamp based on a time of receipt of the first synchronization signal;
receiving, by the first computing device, a synchronization request comprising a second timestamp;
determining, by the first computing device, a synchronized time offset based on the first timestamp and the second timestamp;
transmitting, by the first computing device, a second synchronization signal;
generating, by the first computing device, a synchronization message comprising a third timestamp based on a time of transmission of the second synchronization signal and the synchronized time offset;
queuing, by the first computing device, the synchronization message during a non-accessible transmission time slot; and
wirelessly transmitting, by the first computing device, the synchronization message to a second computing device during an available signal transmission time slot.

2. The method of claim 1, further comprising: receiving, by the first computing device, a synchronization acknowledgment signal from the second computing device.

3. The method of claim 1, further comprising:
receiving, by the second computing device, the second synchronization signal;
recording, by the second computing device, a fourth timestamp based on a time of receipt of the second synchronization signal;
receiving, by the second computing device, the synchronization message; and
determining, by the second computing device, a second synchronized time offset based on the synchronization message and the fourth timestamp.

4. The method of claim 3, wherein the synchronization message further comprises a data acquisition start time, and the method further comprises:
starting, by the second computing device and based on the second synchronized time offset, synchronized data transmission at the data acquisition start time;
receiving, by the first computing device, a synchronized data transmission from the second computing device; and
transmitting, by the first computing device, the synchronized data transmission to a third computing device.

5. The method of claim 1, wherein wirelessly transmitting, by the first computing device, comprises wirelessly transmitting, by the first computing device, the synchronization message via a near field magnetic induction communications link.

6. The method of claim 1, further comprising:
configuring a short range communications link between the first computing device and a third computing device to generate the first synchronization signal during an expected time window;
transmitting the first synchronization signal by a third computing device;
determining, by the third computing device, that the first synchronization signal did not occur during the expected time window;
generating, by the third computing device, a virtual synchronization signal in place of the first synchronization signal; and
transmitting, by the third computing device, the virtual synchronization signal to the first computing device during the expected time window.

7. The method of claim 6 wherein the expected time window comprises a transmission time slot of the short range communications link.

8. The method of claim 1 wherein determining the synchronized time offset comprises:
storing, into memory of the first computing device, a time delay based on the first timestamp and the second timestamp; and
determining, by the first computing device, the synchronized time offset based on the time delay and previously stored time delays.

9. The method of claim 1, further comprising:
receiving, by the first computing device, a fresh synchronization signal after receiving the synchronization request,
wherein queuing the synchronization message occurs after receiving the fresh synchronization signal, and
wherein wirelessly transmitting the synchronization message occurs in the next available signal transmission time slot following receiving the fresh synchronization signal.

10. An implantable system, comprising:
one or more implantable devices comprising:
an implantable substrate comprising a sensor configured to detect and transmit an electromyography (EMG) signal generated by a muscle of a subject, wherein the implantable substrate is in contact with the muscle of the subject; and
a first processor operatively coupled with the implantable substrate and configured to perform operations comprising:
receiving a synchronization signal;
recording a timestamp based on a time of receipt of the synchronization signal;
receiving a synchronization message; and
determining a synchronized time offset based on the synchronization message and the timestamp;
receiving the EMG signal from the sensor;
starting transmission of the EMG signal based on the synchronization message; and
wirelessly transmitting the EMG signal;
one or more wearable devices configured to be attached to the subject, the one or more wearable devices comprising:
a second processor configured to perform operations comprising:
receiving a second synchronization signal from an external device;
recording a second timestamp based on a time of receipt of the second synchronization signal;
receiving a synchronization request comprising a third timestamp;
determining a second synchronized time offset based on the second timestamp and the third timestamp;
transmitting the synchronization signal;
generating the synchronization message comprising a fourth timestamp based on a time of transmission of the synchronization signal and the second synchronized time offset;
queuing the synchronization message during a non-accessible transmission time slot; and
wirelessly transmitting the synchronization message to the first processor during an available signal transmission time slot;
a self-contained battery; and
a power transmitter configured to wirelessly transmit energy to the one or more implantable devices via an inductive magnetic field.

11. The implantable system of claim 10, wherein the first processor is configured to perform operations further comprising:
wirelessly transmitting a synchronization acknowledgement signal; and
wherein the second processor is configured to perform operations further comprising:
wirelessly receiving the synchronization acknowledgement from the first processor; and
wirelessly transmitting the synchronization acknowledgement signal to the external device.

12. The implantable system of claim 10, wherein the synchronization message comprises a time at which to start transmission of the EMG signal.

13. The implantable system of claim 10, wherein the second processor is configured to perform receiving the synchronization request from the external device and transmitting the EMG signal to the external device via a short range communications link.

14. The implantable system of claim 10, wherein the second processor is configured to transmit the synchronization message to the first processor and receive the EMG signal from the first processor via a near field magnetic induction communications link.

15. The implantable system of claim 10, wherein the implantable system is used in a prosthetic limb, an orthotic, or an exoskeleton.

16. A method of controlling a prosthesis, the method comprising:
receiving, by two or more wearable devices a first synchronization signal from an external device, each wearable device associated with an implantable device;
by each wearable device:
recording a first timestamp based on a time of receipt of the first synchronization signal;
receiving a synchronization request from the external device comprising a second timestamp;
determining a synchronized time offset based on the first timestamp and the second timestamp;
transmitting a second synchronization signal to the associated implantable device;
generating a synchronization message comprising a third timestamp based on a time of transmission of the second synchronization signal and the synchronized time offset;
queuing the synchronization message during a non-accessible transmission time slot;
wirelessly transmitting the synchronization message to a second computing device during an available signal transmission time slot;
wirelessly receiving synchronized EMG signals from the implantable devices, the EMG signals being synchronized based on the synchronization message;
wirelessly transmitting the synchronized EMG signals to the external device;
processing the synchronized EMG signals using one or more machine learning classifiers; and
generating a control output for the prosthesis based on the processing.

17. The method of claim 16, further comprising:
configuring a short range communications link between the external device and the wearable devices to generate the first synchronization signal during an expected time window;
transmitting the first synchronization signal by the external device;
determining, by the external device, that the first synchronization signal did not occur during the expected time window;
generating, by the external device, a virtual synchronization signal in place of the first synchronization signal; and
transmitting, by the external computing device, the virtual synchronization signal to the wearable devices during the expected time window.

18. The method of claim 17 wherein the expected time window comprises a transmission time slot of the short range communications link.

19. The method of claim 16 wherein determining the synchronized time offset comprises:
storing, into memory of each wearable device, a time delay based on the first timestamp and the second timestamp; and
determining, by the wearable device, the synchronized time offset based on the time delay and previously stored time delays.

20. The method of claim 16, further comprising:
receiving, by each wearable device, a fresh synchronization signal after receiving the synchronization request,
wherein queuing the synchronization message occurs after receiving the fresh synchronization signal, and
wherein wirelessly transmitting the synchronization message occurs in the next available signal transmission time slot following receiving the fresh synchronization signal.
